(19) United States
(12) Patent Application Publication
(10) Pub. No.: US 2024/0402144 A1
(43) Pub. Date: Dec. 5, 2024

(54) Sensor

(71) Applicant: Croptide Ltd, Hastings (NZ)

(72) Inventors: Daniel James Bloomer, Hastings (NZ); Finn Fitzhugh Brown, Hastings (NZ); Hamish Robert Penny, Hastings (NZ)

(73) Assignee: Croptide Ltd, Hastings (NZ)

(21) Appl. No.: 18/721,159

(22) PCT Filed: Dec. 20, 2022
(86) PCT No.: PCT/NZ2022/050175

§ 371 Date: Jun. 17, 2024

(30) Foreign Application Priority Data
Dec. 20, 2021 (NZ) 783725
Dec. 21, 2021 (NZ) 783780

(51) Int. Cl. G01N 33/00; G01K 1/14; G01K 15/00; G01N 27/22; G01R 27/26

(52) U.S. Cl. CPC G01N 33/0098; G01K 1/14; G01K 15/005; G01N 27/226; G01R 27/2676

(57) ABSTRACT

The present invention relates generally to devices, methods and systems for acquiring plant data. Disclosed is a field-deployable sensor attachable to plants for acquisition of plant-related data comprising: a probe including; a probe body with at least one aperture therethrough which locates the placement and spacing of one or more thermally conductive pins within the tissue of a plant; the one or more thermally conductive pins having distal and proximal ends, said distal end traversing through said aperture in the probe body and insertable into the tissue of the plant, and said proximal end located within the deployable sensor; such that when inserted into the tissue the pin approaches thermal equilibrium with the temperature of the region of the tissue in which the pin is located; and, a temperature sensor (T-sensor) enclosed within the deployable sensor located adjacent the proximal end of the pin to acquire the temperature of the proximal end of the pin; such that the T-sensor through the thermal equilibration of the pin with the plant tissue acquires plant tissue temperature data.

### CROSS REFERENCE TO RELATED APPLICATIONS

This application is a national stage of PCT Patent Application No. PCT/NZ2022/050175, filed Dec. 20, 2022, which claims priority to New Zealand Patent Application No. 783725, filed Dec. 20, 2021, and to New Zealand Patent Application No. 783780, filed Dec. 21, 2021, the disclosures of which are incorporated by reference herein in their entirety.

### TECHNICAL FIELD

The present invention relates generally to devices, methods and systems for acquiring plant data and in particular to field-deployable sensors attachable to plants for acquisition of plant-related data.

### BACKGROUND OF THE INVENTION

Any discussion of the prior art throughout the specification should in no way be considered as an admission that such prior art is widely known or forms part of the common general knowledge in the field.

A sensor is a device that detects and responds to some type of input from the physical environment. A sensor converts a physical phenomenon into a measurable signal such as an analogue voltage or digital signal which can be then transmitted for reading or further processing.

Bioelectrical impedance analysis is a commonly used approach for body composition measurements and assessment of clinical conditions for humans. Bioelectrical impedance information is derived from application of a small (low current and voltage) sinusoidal excitation signal to the biological tissue under investigation.

An example of bioelectrical impedance analysis is Electrical Impedance Spectroscopy (EIS). Typically, when employing EIS, impedance is measured (comprised of real and imaginary parts); an electrical circuit model is formulated for the system, and the parameters then estimated.

Compared to its clinical uses, the use of bioelectrical impedance analysis in the assessment of plant health status has found relatively limited application. However, real-time data acquisition providing information about plant health status is highly desired, within industries such as horticulture and forestry, as it allows ongoing system (e.g. orchard) observation as well as informing critical decision making.

Water scarcity occurs when demand for freshwater exceeds supply in a specified domain. Of all economic sectors, agriculture is the sector where water scarcity has the greatest relevance. It has been stated that agriculture accounts for 70 percent of global freshwater withdrawals, and more than 90 percent of its consumptive use. Under the joint pressure of population growth and changes in dietary habits, food consumption is increasing in most regions of the world (Coping with water scarcity, 2008).

Plant water status is one of the indicators that governs plant physiology, controlling photosynthesis, growth, and productivity, but it can be difficult to measure and monitor. Plant growth rates may be affected prior to observable symptoms. As such, understanding of plant water status can lead to more efficient application of water. Water stress adversely impacts many aspects of the physiology of plants, especially photosynthetic capacity. If the stress is prolonged, plant growth, and productivity are severely diminished (Yuriko Osakabe et al, 2014).

The drought stress responses of vascular plants are complex regulatory mechanisms because they include various physiological responses from signal perception under water deficit conditions to the acquisition of drought stress resistance at the whole-plant level (Fuminori Takahashi et al 2020).

With better water management, growers produce higher quality, high value crops which results in more efficient, and less, water use. While various technologies are available to provide plant water and nutrient status, they are manual, slow, and costly.

Sensors currently used in agricultural systems are mainly focused on the surrounding environment of the plants. Sensors of this kind include sensors that monitor: environmental conditions; air humidity; lighting (sunlight, irradiance); soil moisture and soil nutrients. Other sensing methods include imaging using, for example, satellite or drone technology. Visible and near I.R. image changes are correlatable to physiological changes in plants.

Bioelectrical impedance measurements are temperature dependent and as such it is desirable that biological tissue temperatures are concomitantly acquired with impedance values.

Also desirable for the acquisition of, stepwise continuous, real-time bioelectrical impedance data, such as for a crop, orchard or vineyard, would be a discrete system that is relatively non-invasive for the plant and is sufficiently robust to withstand the at times harsh conditions encountered within the orchard or vineyard such as might be imposed by mechanized activity or weather events.

Options, or alternatives, for apparatus and methods for the relatively non-destructive, real time “continuous step-wise” temperature and bioelectrical impedance data acquisition, allowing for an automated way to measure plant health status, such as water stress, are desirable.

### SUMMARY OF THE INVENTION

In an aspect the invention provides a field-deployable sensor attachable to plants for acquisition of plant-related data comprising: a probe including; a probe body with at least one aperture therethrough which locate the placement and spacing of one or more thermally conductive pins within the tissue of a plant; one or more thermally conductive pins having distal and proximal ends, said distal end traversing through said aperture in the probe body and insertable into the tissue of the plant, and said proximal end located within the deployable sensor; such that when inserted into the tissue the pin approaches thermal equilibrium with the temperature of the region of the tissue in which the pin is located; and a temperature sensor (T-sensor) enclosed within the deployable sensor located adjacent the proximal end of the pin to acquire the temperature of the proximal end of the pin; such that the T-sensor through the thermal equilibration of the pin with the plant tissue acquires plant tissue temperature data.

Suitably, the tissue forms part of a stem, trunk, bole, bough, branch, branchlet, twig, sprig or shoot of a plant.

In embodiments, the at least one aperture further comprises an expanded section forming a well around the thermally conductive pin. In embodiments, the T-sensor is situated within the deployable sensor to record the temperature within the well. Suitably, the well contains a heat transfer media such that when the one or more pins are inserted into plant tissue, the heat transfer between the tissue, pins, and T-sensor establish a thermal equilibrium. Changes in the thermal equilibrium in response to changing environmental conditions affecting plant tissue temperature may be recordable.

In embodiments, the sensor comprises upper and lower thermally conductive pins each with independent T-sensors such that temperature gradients across the tissue into which the respective pins are located are acquirable.

In embodiments, the one or more thermally conductive pins of the deployable sensor are one or more electrically conductive electrodes. The sensor may comprise: an electrical communication means for transmitting electrical data between electrodes and a control system whereby the electrical communication means provides electrical continuity between the electrodes and the circuitry of the control system; and a control system housed within a protective enclosure, including: a printed circuit board (PCB) containing control system circuitry; an analogue front-end for transmitting excitation signals to the electrodes and for converting electrical data received from the electrodes into electrical impedance values; and a microcontroller for programming the analogue front end, and for storing and transmitting data.

Suitably, the deployable sensor has a T-sensor on the PCB for acquiring temperature data within the deployable sensor in the region of the PCB located T-sensor.

Suitably, the sensor comprises a first spaced pair of electrodes. Suitably, the deployable sensor comprises a second spaced pair of electrodes. The first spaced pair of electrodes may be adapted for applying current to the vascular plant tissue and the second spaced pair of electrodes may be adapted for concomitantly measuring voltage. The first spaced pair of electrodes may be an outer pair of electrodes adapted to apply a current signal from the analogue front-end to the vascular tissue and the second spaced pair of electrodes may be an inner pair of electrodes adapted to measure voltage across the system.

Current applied by the outer pair of electrodes to the plant vascular tissue may be variable so as to obtain a constant voltage across the inner pair of electrodes.

The spacing between the pair of inner electrodes may be from about 20 mm to about 80 mm, more preferably from about 40-70 mm and the spacing between the outer pair of electrodes many be from about 60 mm to about 100 mm.

Suitably, the pin shafts comprise an expanded portion of greater cross-sectional area with a lower shoulder that engages with and is retained by a shelf in the probe body, and an upper shoulder which engages with and is retained against the PCB said upper engagement forming electrical communication between the pin and the control system circuitry.

Suitably, the engaging of an electrode shaft with the PCB secures the PCB and the electrode to the probe body.

The protective housing of the sensor may comprise a base and an upper casing. Suitably, the base may be the probe body. The probe body may engage with the upper casing to enclose the control system.

The upper casing may be removably securable against the probe body and when secured against the probe body may retain the electrode heads against complementary electrical communication means.

In some embodiments, the upper casing slidably engages with the probe body to secure the control system against the probe body and the electrode heads against the electrical communication means.

In embodiments, the probe body cooperates with the control system housing to retain the sensor against the plant dermal tissue.

Suitably, the one or more pins have a diameter in the range from about 500 μm to about 2 mm, preferably from about 1 mm to about 2 mm.

In a further aspect the invention provides method for acquiring plant data with a field-deployable sensor attachable to plants comprising: attaching a probe body of the sensor to the plant by inserting one or more thermally conductive pins into the plant tissue wherein said pins traverse through the probe body and are spaced, relatively located, and retained by the probe body; allowing the pins to approach thermal equilibrium with the plant tissue; acquiring stem temperature data with a temperature sensor located adjacent a proximal end of a pin in the probe body, wherein said proximal end approaches thermal equilibrium with the stem tissue.

In embodiments, the pins are electrodes and are in electrical communication with a control system housed in a protective casing said control system including: a printed circuit board; an analogue front-end for transmitting excitation signals to the probe and for converting electrical data received from the probe into electrical impedance values; and a microcontroller for programming the analogue front end, storing and transmitting data; and the method further comprises the steps of: transmitting an electrical excitation signal to the vascular plant tissue through a pair of electrodes; and, acquiring frequency specific electrical impedance values from the plant tissue.

Preferably, the pins are inserted into the tissue of the plant so as to contact extracellular fluid, vascular tissue and ground tissue of the plant.

Preferably, the electrodes comprise a first spaced outer pair and a second spaced inner pair of electrodes and the method further comprises the steps of: a timed application of a current signal to the tissue with the first spaced pair of electrodes with the analogue front-end; simultaneous measurement of the voltage across the tissue with the second spaced pair of electrodes; simultaneous recording of the temperature in the thermal well with a temperature sensor; and acquisition and local storing of the resultant data by the microprocessor.

Suitably, the current applied by the outer pair of electrodes to the plant tissue is varied so as to obtain a constant voltage across the inner pair of electrodes.

Suitably, data recorded by the microprocessor is transmitted to a server wherein the transmitted data includes: unique sensor identification, date, time, temperature and plant tissue electrical data.

The temperature sensors in the field deployable sensor may be calibrated with a calibration means before deployment.

The analogue front-end may be calibrated at one or more frequencies with a calibration means.

In a further aspect the invention provides a field-deployable sensor attachable to plants for acquisition of plant electrical impedance values the sensor including: a probe comprising; a probe body with a non-conductive base with apertures therethrough which locate the placement and spacing of electrodes within tissue of a plant stem; at least a first spaced pair of electrodes, said electrodes traversing through apertures in the probe body and insertable into the tissue of the plant, wherein the first pair of electrodes are at least partially retained by the probe body; electrical communication means for transmitting electrical data between electrodes and a control system contained within the sensor whereby the electrical communication means provides electrical continuity between the electrodes and the circuitry of the control system; a control system housed within a protective enclosure including: a printed circuit board (PCB); an analogue front-end for transmitting excitation signals to the electrodes and for converting electrical data received from the electrodes into electrical impedance values; and a microcontroller for programming the analogue front end and for storing and transmitting data; wherein the probe body cooperates with the control system housing to retain the control system against the stem of a plant.

Suitably, the probe further comprises at least a second spaced pair of electrodes.

In embodiments, the first spaced pair of electrodes is adapted for applying current to the plant tissue and the second spaced pair of electrodes is adapted for concomitantly measuring voltage.

Suitably, the electrode shafts comprise an enlarged portion with a lower shoulder that engages with and is retained by a shelf in the probe body, and an upper shoulder which engages with and is retained against the PCB said upper engagement forming electrical communication between an electrode and the control system circuitry.

In some embodiments, the engaging of an electrode shaft with the PCB secures the PCB and the electrode to the probe body.

The protective housing may comprise a base and an upper casing.

The base may constitute the probe body and engages with the upper casing to enclose the control system.

In some embodiments, the at least one aperture through the probe body comprises an expanded section forming a well.

In yet a further aspect the invention provides a field-deployable sensor attachable to plants for acquisition of plant electrical impedance values, wherein the sensor includes: a probe comprising; a probe body with apertures therethrough which locate the placement and spacing of electrodes within tissue of a plant stem; at least a first spaced pair of electrodes, said electrodes traversing through apertures in the probe body and insertable into the tissue of the plant, wherein the first pair of electrodes are at least partially retained by the probe body; electrical communication means for transmitting electrical data between electrodes and a control system contained within the sensor whereby the electrical communication means provides electrical continuity between the electrodes and the circuitry of the control system; a control system housed within a protective enclosure including: a printed circuit board (PCB); an analogue front-end for transmitting excitation signals to the electrodes and for converting electrical data received from the electrodes into electrical impedance values; and a microcontroller for programming the analogue front end and for storing and transmitting data; and, a temperature sensor housed within the protective enclosure; wherein the temperature sensor acquires temperature data for calculation of electrical impedance values.

### BRIEF DESCRIPTION OF DRAWINGS

The accompanying drawings, together with the detailed description provided below, describe exemplary embodiments of the claimed invention. Like reference numerals refer to like elements throughout the various views of the drawings. The drawings are not to scale and the proportion of certain elements may be exaggerated for the purpose of illustration. The invention will now be described, by way of example, with reference to the accompanying drawings, in which:

FIG. 1 illustrates an isometric view of an embodiment of the present invention;

FIG. 1A illustrates a cross-sectional view of an embodiment of the present invention;

FIG. 2 illustrates an exploded view of an embodiment of the present invention;

FIG. 3 illustrates a side elevation view of a control system of the present invention including partial internal details of the enclosure;

FIG. 4 illustrates a side elevation view of an embodiment of the present invention including partial identification of internal details of the enclosure;

FIG. 4A illustrates an enlarged view of selected region “B” indicated by the circle in FIG. 4;

FIG. 5 illustrates an exploded side elevation view of an embodiment of the present invention;

FIG. 5A illustrates an exploded plan view of an embodiment of the present invention;

FIG. 5B illustrates an exploded isometric view of an embodiment of the present invention;

FIG. 6 illustrates a probe pattern in a stem of a plant;

FIG. 7 illustrates a detailed view of an internal temperature probe of an embodiment of the present invention;

FIG. 8 illustrates a side elevation view of an embodiment of the present invention;

FIG. 8A illustrates an isometric view of an embodiment of the present invention;

FIG. 8B illustrates an enlarged isometric view of the selected region “A” indicated by the circle in the embodiment of FIG. 8A;

FIG. 9 illustrates a side elevation view of an embodiment of the present invention including identification of some internal details;

FIG. 9A illustrates an enlarged view of the selected region “C” indicated by the circle in the embodiment of FIG. 9;

FIG. 10 illustrates a side elevation view of an embodiment of the present invention;

FIG. 11 illustrates a front elevation view of an embodiment of the present invention;

FIG. 12 illustrates an isometric view of an embodiment of the present invention;

FIG. 12A illustrates an exploded view of an embodiment of the present invention;

FIG. 13 illustrates a cross-sectional view of an embodiment of the present invention;

FIG. 13A illustrates an enlarged view of selected region “D” indicated by the circle in the embodiment of FIG. 13;

FIG. 14 illustrates a front elevation view of an embodiment of the present invention;

FIG. 14A illustrates a cross-sectional view of the embodiment of FIG. 14 at the section “O-O” of FIG. 14;

FIG. 15 illustrates an isometric view of an embodiment of the present invention;

FIG. 15A illustrates an exploded isometric view of an embodiment of the present invention;

FIG. 16 illustrates an embodiment of the present invention with the upper casing removed exposing inner details;

FIG. 16A illustrates a cross-sectional view of the embodiment of FIG. 16 at the section “K-K” of FIG. 16;

FIG. 16B illustrates an enlarged view of the selected region “E” indicated by the circle in the embodiment of FIG. 16A;

FIG. 17 illustrates a system of the present invention;

FIG. 18 illustrates a calibration sequence of the present invention; and,

FIG. 19 illustrates a measurement sequence of the present invention.

### DETAILED DESCRIPTION OF THE INVENTION

As a preliminary matter, it will be readily understood by one having ordinary skill in the relevant art that the present disclosure has broad utility and application.

The foregoing summary and following detailed description are merely exemplary in nature and are not intended to limit the described embodiments or the application and uses of the described embodiments. All of the exemplary implementations below are provided to enable persons skilled in the art to make or use the embodiments of the disclosure and are not intended to limit the scope of the disclosure. Furthermore, there is no intention to be bound by any expressed or implied theory presented in the preceding technical field, background, summary or the following detailed description. It is also to be understood that the specific apparatus, systems and processes illustrated in the attached drawings, and described in the following specification, are simply exemplary embodiments of the inventive concept. Specific dimensions and other physical characteristics relating to the embodiments disclosed herein are therefore not to be considered as limiting unless the claims expressly state otherwise.

Any sequences(s) and/or temporal order of steps of various processes or methods that are described herein are illustrative and not restrictive. Accordingly, it should be understood that although steps of various processes or methods may be shown and described as being in a sequence or temporal order, the steps of any such processes or methods are not limited to being carried out in any particular order or sequence, absent any indication otherwise. Indeed, the steps in such processes or methods generally may be carried out in various different sequences and orders while still falling within the scope of the present disclosure.

Additionally, it is important to note that each term used herein refers to that which an ordinary artisan would understand such term to mean based on the contextual use of such term herein. To the extent that the meaning of the term used herein—as understood by the ordinary artisan based on the contextual use of such term—differs in any way from any particular dictionary definition of such term, it is intended that the meaning of the term as understood by the ordinary artisan should prevail.

Furthermore, it is important to note that as used herein “a” and “an” generally denote “at least one” but does not exclude a plurality unless the contextual use dictates otherwise. When used herein to join a list of items, “or” denotes “at least one of the items”, but does not exclude a plurality of items of the list. Finally, when used herein to join a list of items, “and” denotes “all of the items of the list”.

The present disclosure contains headers. It should be understood that these headers are used as references and are not to be construed as limiting upon the subject matter disclosed under the header.

Reference to “the field” is understood to mean reference to work or study that is done in a real, natural environment rather than in a theoretical way or in controlled conditions. In “the field” also implies direct contact with a source of data or subject of interest. In the present context, the field relates to the environment in which a sensor or sensors are deployed, which may be, for example: an orchard, forest, vineyard, or other agricultural environment. The term “field” also has a well-understood meaning in an agricultural context, for example a “field of corn”.

The term “deployable” means able or capable of being deployed. In a broad sense, the term “deploy” can be understood to mean the use of something or someone, especially in order to achieve a particular effect.

As used herein the term “bark” refers to the outermost layers of stems and roots of woody plants. Plants with bark include trees, woody vines, and shrubs. Bark refers to all the tissues outside the vascular cambium and is a non-technical term. Bark overlays the wood and consists of the inner bark and the outer bark. The inner bark, which in older stems is living tissue, includes the innermost layer of the periderm. The outer bark on older stems includes the dead tissue on the surface of the stems, along with parts of the outermost periderm and all the tissues on the outer side of the periderm.

As used herein the term “dermal tissue” refers to the outer protective layer of the primary plant body (the roots, stems, leaves, flowers, fruits, and seeds). Also referred to as the “epidermis” which is a single layer of cells that covers the leaves, flowers, roots and stems of plants. It forms a boundary between the plant and the external environment. The epidermis serves several functions: it protects against water loss, regulate gas exchange, secretes metabolic compounds, and (especially in roots) absorbs water and mineral nutrients. The epidermis is usually one cell layer thick, and its cells lack chloroplasts.

As used herein the term “ground tissue” or “fundamental” tissue refers to tissues that are neither dermal nor vascular. Ground tissue can be divided into three types of plant cells based on the nature of the cell walls: parenchyma, sclerenchyma, and collenchyma. Parenchyma is composed of living cells with a thin cell wall; metabolically active parenchyma cells have thin primary walls and usually remain alive after they become mature. Parenchyma forms the “filler” tissue in the soft parts of plants, and is usually present in cortex, pericycle, pith, and medullary rays in primary stem and root. In botany (plant biology), parenchyma is the simple permanent ground tissues that form the bulk of the plant tissues, such as the soft part of leaves, fruit pulp, and other plant organs. Collenchyma is composed of living cells with a thicker cell wall than parenchyma. Collenchyma cells have thin primary walls with some areas of secondary thickening. Collenchyma provides extra mechanical and structural support, particularly in regions of new growth. Sclerenchyma is composed of dead cells with a thick cell wall (due to an additional cell wall layer referred to as “secondary wall”); primarily for structural support. Sclerenchyma cells have thick lignified secondary walls and often die when mature. Sclerenchyma provides the main structural support to a plant.

As used herein the term “woody plant” refers to a plant that produces wood as its structural tissue and thus has a hard stem. As used herein the term “herbaceous plant” refers to a vascular plant that has no persistent woody stems above ground. Woody plants are perennial plants that have stems that live for several years, adding new growth (height and width) each year. Herbaceous plants have stems that die back to the ground each year. Herbaceous plants may be annual, perennial or biennial.

A number of terms may be used to describe the different anatomical parts of a plant. Vascular plants are broadly comprised of roots, stems and leaves. As used herein the term “stem” includes reference to the main body or stalk of a plant or shrub, typically rising above ground but occasionally subterranean. In botany, the trunk (or bole) is the stem and main wooden axis of a tree. The terms trunk, bole and stem are frequently used interchangeably. Other types of plant stems include: boughs, which are large divisions of the main stem axis, branches, which are divisions of the main axis of the stem or another branch, branchlets, twigs, sprigs and shoots.

Plant biological tissue is made up of cells including a cell (plasma) membrane, cell walls and fluid within and outside of cells, for example, sap. The different components of the tissue demonstrate differences in electrical conductivity. That is, the tissues consist of conductive and non-conductive elements with fluid volumes, tissue properties and cell membranes as the primary electronically recognised constituents. As used herein the term “plasma membrane” (also called the cell membrane), refers to the membrane found in all cells that separates the interior of the cell from the outside environment. In bacterial and plant cells, a cell wall is attached to the plasma membrane on its outside surface. The plasma membrane consists of a lipid bilayer that is semipermeable. The plasma membrane regulates the transport of materials entering and exiting the cell. The lipid bilayer forms an insulating barrier separating salt solutions of the intercellular fluid (ICF) from the extracellular fluid (ECF). When an organism becomes a component of a safe and highly controlled electrical circuit, the measured change (decrease) in voltage following the administration of a radiofrequency alternating current yields bioelectrical measurements that designate structural and functional biological variables. The extracellular space (ECS or apoplast) is the plant cell compartment external to the plasma membrane, which includes the cell walls, the intercellular space and the apoplastic fluid (APF). An electrical capacitor is a charge-storing device, which consists of two conducting plates separated by an insulating barrier. Because the lipid bilayer of the plasma membrane forms an insulating barrier separating the electrically conductive salt solutions of the ICF and ECF, the plasma membrane behaves as a capacitor.

As used herein the term “apoplastic” and related terms such as “apoplast”, refer to the intercellular space (also known as “extracellular space”) filled with gas and water contained between cell membranes.

As used herein the term “symplastic”, and related terms such as “symplast”, refer to the inner side of the plasma membrane of a plant in which water and low-molecular weight solutes can freely diffuse.

As used herein the term “extracellular fluid” refers to fluid that is located between cells (intercellular), and not contained in cells.

As used herein the term “intracellular fluid” refers to fluid that is located or occurring within a cell or cells.

As used herein the term “water potential” refers to the potential energy of water per unit volume relative to pure water in reference conditions. The water potential quantifies the tendency of water to move from one area to another due to osmosis, gravity, mechanical pressure, capillary action, matrix effects. The addition of solutes, such as dissolved salts, typically lowers the potential, whereas an increase in pressure or elevation will raise the water potential. With no restriction to flow, water will move from a region of high-water potential to a region of lower water potential (pure water has a reference potential of zero). Water potential, therefore, may be used to indicate the demand for water within the plant, the resistance to water movement within the plant, and water loss by transpiration to dissipate heat and cool the leaves.

As used herein the term “transpiration” refers to the process of water moving through a plant and its evaporation from aerial parts such as leaves, stems and flowers. As used herein the term “evapotranspiration” refers to the sum of evaporation from the land surface and transpiration from plants.

As used herein the term “plant health status” refers to the physical (anatomic or phytotomic), physiologic, and biochemical status of a plant. Measures of health status include: nutrients (e.g. availability or deficit), water stress and disease. For example, an indicator of plant health is stomatal activity, which: is affected by environmental stresses, can influence CO₂ absorption, and thus impact photosynthesis and plant growth. In response to a water deficit stress, ion- and water-transport systems across membranes function to control turgor pressure changes in guard cells and stimulate stomatal closure (T. F. Döring, et al, 2012).

As used herein the term “vascular plant” (also known as a tracheophyte) refers to a large group of land plants with lignified tissues (the xylem) for conducting water and minerals throughout the plant. They also have a specialized non-lignified tissue (the phloem) to conduct products of photosynthesis. As used herein the term “vascular tissue” refers to a complex conducting tissue, formed of more than one cell type, found in vascular plants. The primary components of vascular tissue are the xylem and phloem.

These two tissues transport fluid and nutrients internally. There are also two meristems associated with vascular tissue: the vascular cambium and the cork cambium. All the vascular tissues within a particular plant together constitute the vascular tissue system of that plant.

As used herein the term “phloem” refers to the vascular tissue in plants which conducts sugars and other metabolic products downwards from the leaves. The phloem conducts food made in leaves to all other parts of the plant.

As used herein the term “xylem” refers to the vascular tissue in plants which conducts water and dissolved nutrients upwards from the root and also helps to form the woody element in the stem. Xylem consists largely of dead cells (parenchyma is the only living cells present in the xylem). The xylem and parenchyma are tracheary elements.

As used herein the terms “vessel element” or “vessel member” (also called trachea or xylem vessel) refers to one of the cell types found in xylem, the water conducting tissue of plants.

As used herein the term analogue front-end (AFE) refers to a semiconductor device that is used for analogue signal conditioning that is comprised of analogue amplifiers, op amps, filters, and sometimes application-specific integrated circuits. This configurable functional block is used to interface with a variety of sensors (to interface sensor with, for example, an antenna, analogue-to-digital converter or, in some cases, to a microcontroller).

As used herein the term “microcontroller” (MCU for microcontroller unit) refers to a small computer on a single metal-oxide-semiconductor (MOS) integrated circuit (IC) chip. A microcontroller contains one or more CPUs (processor cores) along with memory and programmable input/output peripherals. Programme memory in the form of ferroelectric RAM, NOR flash or OTP ROM is also often included on chip, as well as a small amount of RAM. Microcontrollers are designed for embedded applications, in contrast to the microprocessors used in personal computers or other general purpose applications consisting of various discrete chips. Microcontrollers are used in automatically controlled products and devices, such as automobile engine control systems, implantable medical devices, remote controls, office machines, appliances, power tools, toys and other embedded systems.

As used herein “applying voltage” refers to applying a “voltage source”. A voltage source is a two-terminal device which can maintain a fixed voltage. An ideal voltage source can maintain the fixed voltage independent of the load resistance or the output current. However, a real-world voltage source cannot supply unlimited current. A voltage source is the dual of a current source. Real-world sources of electrical energy, such as batteries and generators, can be modelled for analysis purposes as a combination of an ideal voltage source and additional combinations of impedance elements.

As used herein the term “applying current” means applying a “current source”. A current source is an electronic circuit that delivers or absorbs an electric current which is independent of the voltage across it. A current source is the dual of a voltage source. The term current sink is sometimes used for sources fed from a negative voltage supply. There are two types of current sources. An independent current source (or sink) delivers a constant current. A dependent current source delivers a current which is proportional to some other voltage or current in the circuit.

As used herein the term “bioimpedance”, and related terms such as “biological impedance” and “bioelectrical impedance”, refer to the ability of biological tissue to impede electric current. Bioimpedance is the opposition to the flow or passage of an alternating current (AC signals) through cellular walls and membranes, and intracellular and extracellular ionic solutions (through a biological electrical system). For example, plant sap has electrical properties due to its ionic content, and plant biological tissues can behave as electrical circuits made with passive elements such as resistors and capacitors which together constitute electrical bioimpedance.

From a purely electrical point of view, impedance (Z), is the obstruction to the flow of an alternating current and, hence, is dependent on the frequency of the applied current, defined in impedance magnitude (|Z|) and phase angle (φ) as shown in Equations (1)-(3). Bioimpedance is a complex quantity composed of resistance (R) which derived from fluid in the biological system water and reactance (Xc) that is caused by the capacitance from structural components, such as cell membranes. Bioimpedance is the sum of resistance and reactance:

$$Z = R + {jX}_{c} \qquad 1.$$

$$\vert Z \vert = \sqrt{{R}^{2} + {{X}_{c}}^{2}} \qquad 2.$$

$$ϕ = {tan}^{- 1} ( \frac{{X}_{c}}{R} ) \qquad 3.$$

Analysis of bioimpedance that is obtained at more than two frequencies is known as multiple-frequency bioimpedance analysis. An electrical impedance measurement is the measurement of complex resistance in the presence of AC current.

As used herein the term “reactance” refers to the opposition of a circuit element to the flow of current due to that element's inductance or capacitance. The reactance represents the delay in the conduction or passage of the administered current by cell membranes and tissue interfaces. Capacitive reactance is the opposition to the change in voltage across an element. Inductive reactance is the opposition to the change of current through an element.

Resistance is a measure of the opposition of something to non-oscillating current passing through it. Reactance is a measure of the opposition of something to an alternating current passing through it, due to the presence of electric or magnetic fields set up by the alternating current. Reactance comprises capacitance and inductance. Capacitance is a measure of the opposition to the current due to accumulated charge, and inductance is a measure of the opposition to the current due to the rate of change of the current. Inductance and capacitance are components of impedance.

Capacitance is a function of reactance (due to membrane structure and function) and causes the current to lag behind the voltage creating a phase shift angle that is quantified as the angular transformation of the ratio of reactance to resistance. Capacitance is a component of electrical impedance. Capacitance is directly proportional to the surface area of the plates and inversely proportional to the distance separating the two plates. In biological circuit equivalents the capacitance depends on the characteristics of the insulating material (lipid bilayer).

The AC signal passage generates amplitude changes and phase shifts between output and input system signals, and is frequency-dependent. The electrical impedance of biological tissues is a variable that can be affected by various factors, such as water level and diseases and may be used as an indicator of plant health status.

It has been found that plant tissue impedance largely depends of three factors between the frequency range of 10 Hz to 1 MHz:
Intracellular (symplastic) resistance;
Intercellular (apoplastic or extracellular) resistance, and;
impedance of the cell membrane.

The bioimpedance of a biological system, such as living vascular plant tissue, is temperature dependent. Generally, the conductivity of a solution increases with temperature, as the mobility of the ions increases. Thus, as, for example, the temperature of extracellular fluid increases, the resistivity of the fluid decreases.

Temperature dependent functional changes to the cell membrane (e.g. opening or closing of channels) may lead to changes in the resistive/capacitive relationship in models representing the lipid bilayer. An example of such a change is as follows: in response to a water deficit stress, ion- and water-transport systems across membranes function to control turgor pressure changes. The above-mentioned water deficit stress could be induced by high temperatures.

As used herein the term “turgor pressure” refers to the force within the cell that pushes the plasma membrane against the cell wall. It is also called hydrostatic pressure, and is defined as the pressure measured by a fluid, measured at a certain point within itself when at equilibrium. Generally, turgor pressure is caused by the osmotic flow of water and occurs in plants, fungi, and bacteria. The phenomenon is also observed in protists that have cell walls. This system is not seen in animal cells, as the absence of a cell wall would cause the cell to lyse when under too much pressure. The pressure exerted by the osmotic flow of water is called turgidity. It is caused by the osmotic flow of water through a selectively permeable membrane. Osmotic flow of water through a semipermeable membrane is when the water travels from an area with a low-solute concentration, to one with a higher-solute concentration. In plants, this entails the water moving from the low concentration solute outside the cell, into the cell's vacuole.

The term “stomata” refers to the minute openings found in the epidermis of leaves, stems and other plant organs. Stomata allow gases such as carbon dioxide, water vapour and oxygen to diffuse into and out of the internal tissues of the plant. Guard cells are specialized plant cells in the epidermis of leaves, stems and other organs that are used to control gas exchange. They are produced in pairs with a gap between them that forms a stomatal pore. The stomatal pores are largest when water is freely available and the guard cells turgid, and closed when water availability is critically low and the guard cells become flaccid. Photosynthesis depends on the diffusion of carbon dioxide (CO₂) from the air through the stomata into the mesophyll tissues. Oxygen (O₂), produced as a by-product of photosynthesis, exits the plant via the stomata. When the stomata are open, water is lost by evaporation and must be replaced via the transpiration stream, with water taken up by the roots. Plants must balance the amount of CO₂ absorbed from the air with the water loss through the stomatal pores, and this is achieved by both active and passive control of guard cell turgor pressure and stomatal pore size.

The temperature of a plant stem follows environmental conditions. Increases in ambient temperature leading to an increase in stem temperature. There is expected to be a lag between ambient temperature changes and stem temperature deriving from, for example, the difference between microclimate temperature and macroclimate temperature, the thickness of the stem and the thermal conductivity of the stem. Other prevailing conditions may also play a role, for example, wind speed.

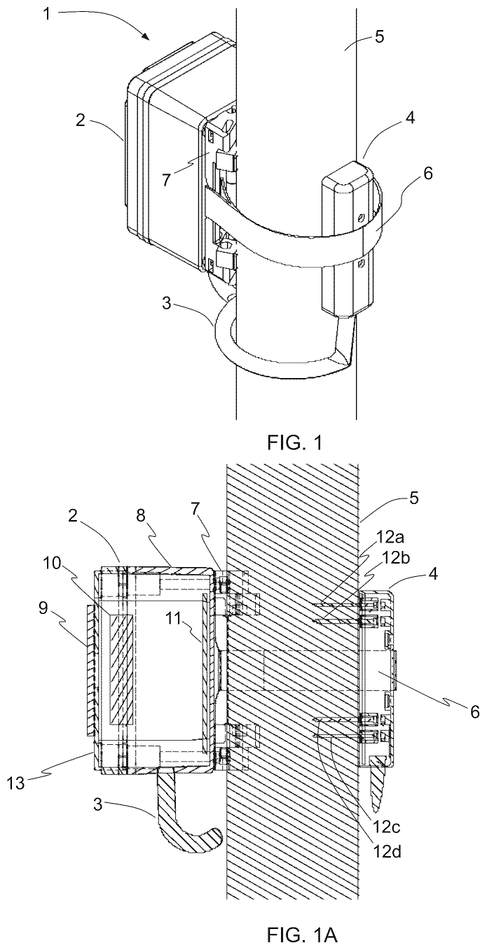

Considering now the drawings, FIG. 1 illustrates an isometric view of an embodiment of the present invention. As displayed in FIG. 1, the field-deployable sensor 1 for acquisition of plant data, includes: a control system enclosure 2 having a protective casing for the electrical circuitry contained therein, a probe 4 which is retained against a stem 5 of the plant by a retaining means 6 and electrical communication means 3 for the transmission of information between the control system and the probe body. The electrical communication means provides electrical continuity between the electrodes and the control system circuitry. The control system enclosure 2 may be of any suitable shape and is preferably of a low profile to minimise undesired contact with the enclosure 2 such as might accidently occur with machinery in an orchard or vineyard. Preferably, the sensor is low profile to minimise possible forces on the probe pins, which could damage the plant and/or affect readings, and to minimise the possibility of accident from machinery such as harvesters or hedgers.

In the embodiment displayed in FIG. 1, the retaining means 6 is attached to at least one bracket 7 which abuts the stem 5 and which is also in turn fastened to the control box 2. It should be understood that the retaining means may attach directly to the control box 2 without the need for bracket 7. The retaining means 6 may be, for example, a strap, such as an elastic strap, to retain both the control system enclosure 2 and probe 3 against a stem 5 of the plant, to which the sensor is attached, and which may be tightened or loosened as required. As illustrated, the body of the probe 4 cooperates with the control system enclosure through the use of a retaining means, in this case a strap, to retain the control system enclosure in place. Opposing forces on the probe and control system housing, imparted by the retaining means, retain the probe and housing against the stem.

It is noted that if unduly tight, the force of the strap may act to constrict the stem, potentially affecting xylem vessels and/or stem growth. This constriction may also have an effect on impedance. Accordingly, the strap 6 may be adjusted to be sufficiently tight so as to sufficiently securely retain the sensor 1 in place on the plant but not so tight that the strap acts to constrict plant function (for example, affect the natural expansion and contraction of the stem 5. Moreover, as the plant grows, and the diameter of the stem 5 increases, a fixed strap will become tighter and the constriction may become more marked. To overcome this, the strap 6 may be gradually loosened over a season as the stem undergoes seasonal growth. As discussed, the constriction effect may be mitigated, to some extent, by adjusting the ‘tightness’ of the strap. However, there is a trade-off between stem 5 constriction and support for the control box 2 and probe 4. An alternative to overcome this problem is use of a mechanical strapping system that gradually releases over time or that may be manually readjusting seasonally.

Probe 4 is typically comprised of an insulating probe body, which locates the placement and spacing of pins within plant tissue, a base which abuts the stem upon insertion of the probe pins into the plant tissue, and conductive pins which traverse through and are at least partially retained by the probe body. The probe body provides thermal insulation and electrical insulation between the pins. The probe body is also comprised of apertures for pins. The pins pass through the probe body and extend from the base. The pins which pass through the apertures are guided and at least in part retained by the probe body or locator. Suitably, the pins are thermally and electrically conductive. The pins are relatively located (in respect of each other and the stem into which they are to be inserted) by the probe body. In use, the distal ends of the pins are inserted in the stem of a plant. The applicants have found that advantageously the pins should be inserted within the stem so as to preferably contact all of the extracellular fluid, vascular tissue and ground tissue of the stem. When acquiring bioelectrical data, it was found that contact with all of the extracellular fluid, vascular tissue and ground tissue of the stem, allows acquisition of readings relating to each type of tissue. It was found that such contact allows measurement where the tissue types are in parallel, rather than having the tissue types in series, with respect to the conductive pathway between the electrodes. In this way a more accurate representation of tissue impedance may be derived.

The pins may be both thermally conductive and electrically conductive. In the latter case the pins act as electrodes. When acting as electrodes, the proximal ends of the electrodes are configured to connect to sensor circuitry so as to enable the establishment of electronic connectivity between the probe and the control system contained within the system enclosure. The distal ends of the electrodes are inserted into plant tissue.

The sensor 1 may be attached to a stem 5 of any plant which is of sufficient diameter, rigidity and strength to support the weight of the sensor 1. The biosensors of the present invention have application with stems of woody plants, including stems with heavy bark covering, such as trunks. Examples include, vines, such as kiwifruit and grape vines, and fruit trees. The sensors of the present invention may also be used with non-woody plants that have sufficient mechanical structure to support the sensor weight and/or plants that grow horizontally across the soil surface. Examples, of non-woody plants include cannabis, tomatoes and cucumbers. Typically, sensors may weigh from about 40 grams to about 200 grams. Preferably, sensors may weigh between about 25 and about 75 grams.

Turning now to FIG. 1A, illustrated is a cross-sectional view of the sensor embodiment illustrated in FIG. 1. Additional features within the control system enclosure 2 are identified in FIG. 1A including: a protective housing 8 to protect the control system components from, for example, environmental conditions and mechanical damage; a rechargeable battery 10 which powers the control system circuitry; a solar panel 9 which recharges the rechargeable battery 10 and which is attached to an access panel 13 the removal of which allows access to the inner contents of enclosure 2; and, one or more printed circuit boards (PCBs) 11 contained within the control box 2 and which contain sensor circuitry. Also illustrated are four electrodes 12a-12d which are shown extending from probe 4 into the stem 5 of the plant. Although the illustrated embodiments show 4 pins, or two pairs of pins, an outer pair 12a and 12d and an inner pair 12b and 12c, it is to be understood that alternative numbers of pairs of electrodes may be suitable for use in certain applications. The pins may also be grouped as an upper pair 12a and 12b and a lower pair 12c and 12d, although it is understood this is arbitrary and mainly of assistance for discursive purposes (for example, the sensor may be turned around to run effectively longitudinally downwards). A tetrapolar electrode configuration may be used, for example, to compensate for cable impedance. A tetrapolar configuration allows for accounting of the intrinsic electrode impedance in the biocircuitry models that may be employed. It is understood that the impedance is typically higher at the electrode/stem interface. A known problem is that low frequency polarisation occurs in ionic systems. A tetrapolar approach also assists in low frequency analysis. The spacing between the electrode pairs may affect the magnitude of the impedance value acquired with greater spacing resulting in a higher magnitude of impedance. It has been observed that greater spacing, and the concomitant increased region of stem tissue between electrodes, leads to more consistent data acquisition. However, it has also been observed that greater spacing increases the likelihood of inconsistencies in the stem material, such as knots or scar tissue, being located in the stem region between the electrodes. These inconsistencies can have a confounding effect on the acquisition of representative impedance values. Suitably, the first pair of electrodes is an inner pair of electrodes and the second pair of electrodes is an outer pair of electrodes.

Suitably, the inner electrode pair is spaced from about 20 mm to about 80 mm, more suitable from about 50 mm to about 70 mm. The locating and spacing probe body, accurately, and reproducibly spaces and locates all electrodes, individually and as pairs with respect to each other. The significance of the reproducibility of accurate spacing and alignment relates to the body and mass of tissue through which electrical signals are passed which varies with spacing and alignment. Suitably, the spacing between the outer pair of electrodes is from about 60 mm to about 100 mm. It was also found that pins the are preferably not to be located too close to one another other as this may result in tissue damage, for example stem splitting, which may act to confound the bioelectrical data acquisition.

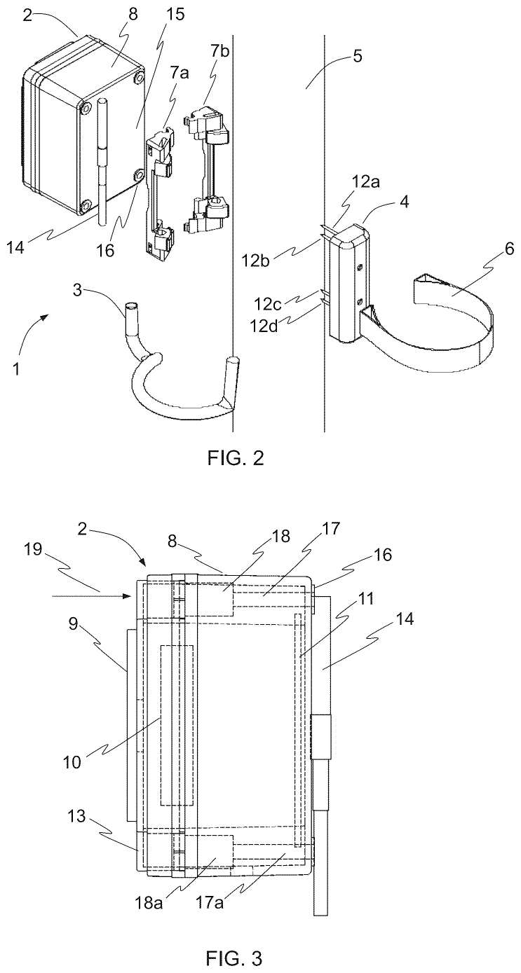

FIG. 2 illustrates an exploded view of an embodiment of the invention. An air temperature sensor 14 is displayed attached to the removable back panel 15 of the control box 2. One or more apertures 16 may be used for the attachment of items ancillary to the control box 2, such as temperature sensors. The one or more apertures 16 may, for example, be configured for use with fasteners such as for fastening the removable back plate 15 to box 2. Also displayed in FIG. 2 are the complementary brackets 7a and 7b which in use are attached to the control box 2 and which, in application, abut both stem 5 and control box 2 and assist in stabilising the control box 2 against the stem 5 by conforming the control box 2 to the dimensions or shape of the stem 5.

Returning to temperature sensor 14, suitably, the air temperature sensor (air T-sensor) 14 may be selected from a thermistor, thermocouple or RTD. Thermocouples are self-powered, require no excitation, can operate over a wide temperature range, and have quick response times. An RTD is a resistor with well-defined resistance vs. temperature characteristics. Platinum is the most common and accurate material used to make RTDs. Thermistors are similar to RTDs in that temperature changes cause measurable resistance changes. Thermistors are usually made from a polymer or ceramic material. In most cases, thermistors are cheaper but are also less accurate than RTDs. Most thermistors are available in two wire configurations.

In some embodiments, a temperature sensor may be located on the surface of the stem such as attached against the bark of the stem. In some embodiments a temperature sensor may be located within the control enclosure 2 such as on a printed circuit board 11 within the housing 8, for example, a semiconductor-based temperature sensor IC such as a local temperature sensor or remote digital temperature sensor. Suitably, the T-sensor on the PCB (PCB T-sensor) is adapted to acquire temperature data within the deployable sensor in the vicinity of the PCB T-sensor.

Turning now to FIG. 3, illustrated is a side elevation view of the control system enclosure 2 containing control circuitry with some internal features within box 2 indicated with hashed lines. As discussed, access to the control system within enclosure 2 may be achieved through removal of the front panel 13 of the box 2. The arrow 19 indicates the location and direction of a screw which engages with screw hub 18 to secure the cover or lid 13 in place. The raised portion 16 indicates the location of an aperture for access to tapped hole 17. Both the bottom and the top panels of the control system enclosure may be removable. In some embodiments a single fastener may be used. Alternatively, multiple fasteners and complementary threaded channels may be employed to secure the lid 13 of the box 2, for example four fasteners located four corners of the control box. Suitably, the tapped channels are plastic tapped holes which are moulded into the interior of the enclosure. Furthermore, the enclosure may have one or more PCB standoffs moulded into the interior of the box for securing PCBs to the enclosure.

The control box enclosure may include one or more knockouts for modification of the control box, if required. Modifications might include, for example, a new cable way inlet to allow for implementation of, for example, a further temperature sensor, or addition of cable for a soil sensor. Suitably, the control box embodies a field-deployable data acquisition hub.

The protective housing 8 of the control box 2, which includes, if present, removable lids or other access coverings, provides protection against environmental conditions such as dust, splashing mud, water from rain or irrigation, temperature and ultraviolet (UV) radiation. The enclosure may be of lap joint construction which provides protection against the access of dust and splashing water. The join between the lid and casing may include a suitable sealing means such as a rubber gasket. An Ingress Protection (IP) rating system is used to define levels of sealing effectiveness of electrical enclosures against intrusion from foreign bodies and moisture. Suitably, the enclosure has an IP rating of IP55-IP66. The control box may be formed from any suitable material such as halogen free, flame-retardant plastic of sufficient mechanical strength to withstand at least some of the accidental impacts that may result from, for example, horticultural, agricultural or forestry activities, such as, mechanised orchard or vineyard activities. An example of a plastic material durable under the environmental conditions described above is acrylonitrile styrene acrylate (ASA). Fibreglass is optionally another material which may be employed.

Preferably, the enclosure is formed from a recycled plastic. Examples of recycled plastic include: polyethylene terephthalate (PET); high density polyethylene (HDPE) and various forms of polypropylene (polypropene) including both homo- and copolymers. A particularly preferred recycled plastic is a recycled polypropylene.

The location of the sensor is preferably in-line with the row of plants. That is, on a north-south oriented row of plants, the sensor is located on the northern or southern side of the stem. This reduces the likelihood of damage to or accidental removal of the biosensor through activity of machinery such as hedgers and harvesters.

Field conditions, such as found in an orchard or vineyard, are subject to natural climatic variations including diurnal temperature swings, wind, and precipitation events such as rain, hail or snow. Swings in temperature may lead to pressure changes within the protective housing—an increase in temperature leading to an increase in pressure within the housing. Expansion (and concomitant escape) of the air within the housing leads to a vacuum forming when the temperature of the housing drops. In the event of a negative pressure within the housing there is the possibility of moisture being drawn into the housing during pressure equalisation. Accordingly, in some embodiments, the protective housing 8 of the control box 2 includes pressure equalising breathers (breather valves—not shown). As used herein the term “breather valve”, also known as a “pressure relief valve”, refers to a valve that prevents excessive pressure or vacuum build-up in sealed containers. An example of a preferred breather valve is a desiccant breather valve which employs a filter media to remove particles and a silica-gel desiccant to remove moisture from the air entering the oil. Suitably, the filter media and silica gel desiccant are replaceable once saturated.

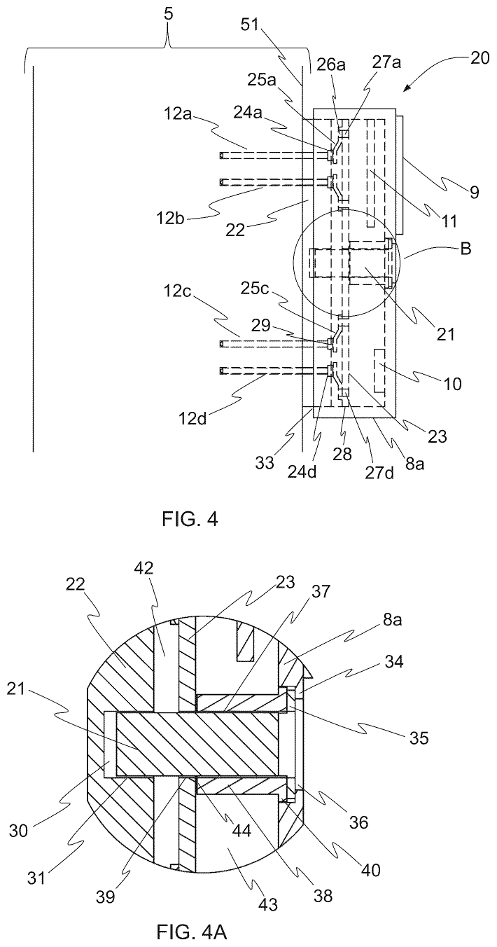

Considering now FIG. 4, illustrated is an alternative embodiment 20 of the present invention wherein the control box containing sensor electronics is attached to the back of the probe—affixed therewith with screw 21 by actuation of screw head 35 (see FIG. 4A). In FIG. 4 the sensor 20 is illustrated as retained in and against the plant stem 5 by the electrodes 12a to 12d, which extend into the tissue of stem 5. In a similar manner to the previously discussed embodiment, sensor 20 is comprised of an electrical control system within an enclosure; electrical communication means between the control system and probe which is comprised of an insulating probe body and pins. In the embodiment of FIG. 4, the probe is constituted of at least one or more pair of electrodes and the probe body acts as a guide or locator for spaced arrangement of the pins within the stem tissue. The electrode pins are formed from a suitably conductive material. The pins are suitably both electrically and thermally conductive. On insertion into the stem the pins are in contact with the biological tissue of the stem. The control system is adapted to send excitation signals to plant tissue through the pins and acquire plant data derived from the excitation signal including bioelectrical data, acquire tissue temperature data, store data, and transmit data.

Copper and copper alloys are suitable conductive materials. However, in some environments, copper may be prone to corrosion. The electrical conductivity of contact materials can be largely reduced by corrosion and in order to avoid corrosion, protective surfaces may be used. There are at least two groups of materials, which can be employed for corrosion protection of electrically conductive surfaces. The first group includes noble metals such as gold, silver and palladium. The second group is comprised of corrosion resistant so-called passive metals such as tin and nickel. These metals are basically ignoble and they derive their corrosion resistance from the presence of a thin oxide film on the surface, called a passive film, which acts as a protective barrier between the metal and its environment [Song et al, 2012]. Preferably the pins are formed from corrosion resistant material.

A suitable electrode material is a duplex which are high chromium stainless steels. Duplex steels are high strength materials which are similar to copper on the galvanic corrosion scale. Duplex stainless steels are highly corrosion resistant. The thermal conductivity for duplex-type stainless steels ranges from about 12 to about 16 W/m·K. It was determined, using ice water baths to test equilibration times, that thermal equilibrium of the electrodes inserted in the water bath to the bath temperature was reached in under 10 minutes.

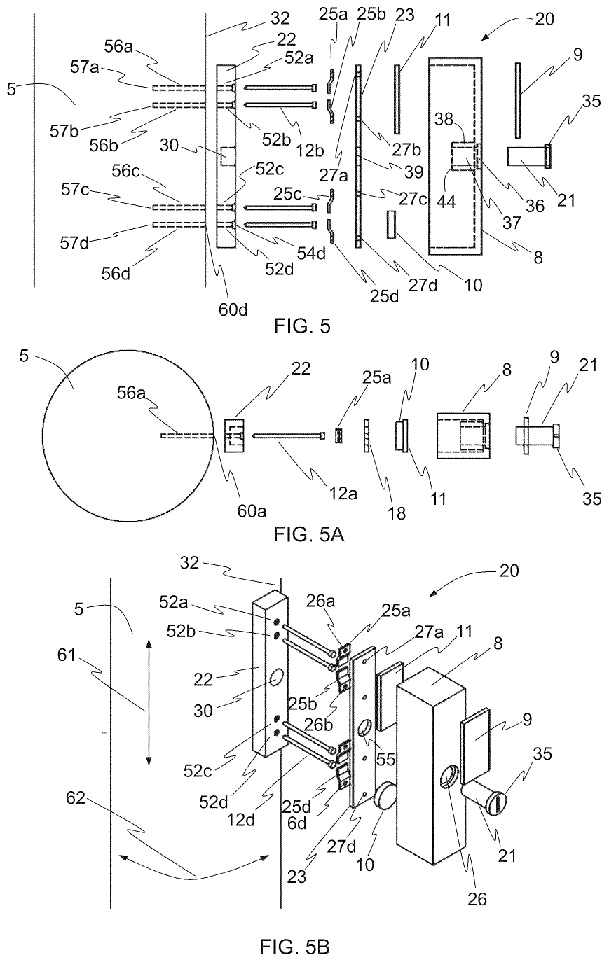

Continuing with FIG. 4, four pins 12a to 12d are shown each of which has a corresponding head 24a to 24d (see also FIGS. 5, 5A and 5B). In the present embodiment the pins are thermally and electrically conductive (electrodes). The corresponding electrode heads 24a-24d are in contact 29 with connectors 25a to 25d (see also FIGS. 5, 5A and 5B). In the embodiment illustrated, each of the connectors 25a to 25d is attached to a backplate 23 so as to align the connectors 25a to 25d with the respective electrode heads 24a to 24d. Not all elements of the embodiment are indicated in the figure, however, considering, for the moment, only electrode 12a, the head 24a of the electrode is in contact with the connector 25a so as to provide electrical communication between the electrode and the connector. An engaging means 26a of the connector is further aligned with a connecting means 27a of the back plate 23. Similar connectivity arrangements may be assumed for pins 12b, 12c and 12d.

The backplate 23 is typically constructed from an electrically insulating material. The connecting means 27a and engaging means 26a may be, for example, connecting apertures. For example, the connector 25a may be attached to the backplate by a fastener, such as a screw, which passes through the aligned apertures 26a and 27a. Suitably, the screw is a formed from a conductive material and may also attach a conductive lug to the connecting means such that a further connection may be made to a PCB within enclosure 2, such as with an electrical conduit. The connecting means 27a may also be a through-hole via or blind via. The connector 26a may be soldered to the connecting means 27a on the probe side and a wire soldered to the connecting means 27a on the control box side. In any event, the connections between the head 24a of the electrode 12a, the connector 26a and the connecting means 27a of backplate 23 allow for discrete electrical communication between the plant tissue in contact with the electrode 12a and the electronic circuitry of the control box. As mentioned, similar connectivity is established for each of the electrodes 12b, 12c and 12d.

Rain and irrigation events may cause moisture to run down the stem surface 32 of the stem 5. The presence of external moisture on the pin shafts has the potential to affect impedance measurements of the stem biological tissue. Unwanted water ingress between the probe and the stem may be exacerbated when attachment is made to woody plants whose outer bark often has an undulating or corrugated surface. Preferably, the locator 22 abuts the stem 5 in close contact with the surface 32 of the stem such that contact of moisture with the electrode pins 12a to 12d is minimised. The surface 32 has a layer of dermal tissue. Suitably, the backplate 23, which partitions the probe from the control system, provides a further moisture seal between the electronic circuitry contained within control system and the probe such as with a moisture sealing engagement 28 between inner wall of the housing 8 and the edge of the backplate 23. Suitably, at least a portion of an electrode, which may come in contact with external moisture, that is the portion which traverses the stem-locator interface, may be coated with a moisture resistant protective resin so as to minimise interference with impedance measurements. Furthermore, the circuitry, and circuit connectors, may be potted or encapsulated, for example with an epoxy resin, to protect from moisture. Thermosetting plastics or silicone rubber gels may also be employed. Additionally, insulating material, such as a moisture insulating foam material may be injected into cavities, for example, cavity 42 or cavity 43 (see FIG. 4A), injected through one or more knockouts in the enclosure. The embodiment of FIG. 4 includes a PCB 11 for control system circuitry, a rechargeable battery 10 for powering the control system, a solar panel 9 for charging the battery.

FIG. 4A illustrates a detailed view of the fastening means which attaches the control box to the probe. The screw 21 extends through channel 37 in housing 38 an aperture 39 the backplate 23 and engages with a threaded 31 hub 30 in the locator 22. The backplate abuts 44 the screw housing 38, which in the present embodiment, is formed integrally with the casing upper casing 8a. The length of the housing 38 defines the location of the backplate within the protective casing 8. In the present embodiment the protective casing 8 extends to enclose 33 (see FIG. 4) at least a portion of the locator 22. The screw 21 may be a captive 34 screw such that on disengagement of the screw with the threaded 31 channel 30, the screw remains captive to the housing 8 and is not, therefore, easily lost. Suitably the screw head 35 are recessed 36 within the upper enclosure 8a of the sensor.

Considering FIGS. 5, 5A and 5B together: FIG. 5 illustrates an exploded side elevation view of the embodiment of FIG. 4, FIG. 5A illustrates an exploded plan view of the same embodiment, and FIG. 5B illustrates an exploded isometric view of the same embodiment. Channels 56a to 56d through which electrodes pass through the locator 22 into the plant stem are shown in FIG. 5. In embodiments, when inserting electrodes into the plant stem, a pilot hole may be first prepared such as with a drill. In embodiments, the electrode pins may be inserted or pushed into the plant tissue without the need for drilling of pilot holes.

It was found that the electrode pins are sufficiently retained within the stem tissue to mechanically support a load such as might derive from the probe and the control box. For example, it was found that the use of four stainless steel nails as electrode pins extending 16 mm from the probe body base in the plant was sufficient to provide mechanical support for the biosensor. Initial testing indicated the degree of support provided will be sufficient to securely retain the sensor against the stem.

In some embodiments, and considering the example of substantially cylindrically shaped electrode pins, the pilot channel 56a to 56d diameter, that is drilled in preparation for insertion of the electrode pin, is slightly less than the diameter of the pin allowing for press fitting of the pin within the stem 5. The woody tissue of the stem compresses slightly as the pin (for example pin 12a as shown in FIG. 5A), is inserted into channel 56a through aperture 60a into stem tissue stem thus providing a tight, retaining fit for the pin which is them sufficiently firmly held within the stem 5 to retain the sensor against the stem.

The pins may have modified heads to assist with retention of the pin within the stem wood. For example, the pin head may have a slight arrow shape or barb feature. Other modifications to the pins to assist with retention of the pin within the stem, and or to alter the surface area of the pin, include ribbing, corrugations or undulations.

The surface area of the electrode pin in contact with the stem tissue may be adjusted through changing the cross-sectional area and or exposed surface area of the pin. For example, in the case of pins with a substantially circular cross-sectional area, the pin diameter may range from about 500 μm to about 3.5 mm, more preferably from about 1 mm to 2.5 mm and more preferably from about 1.2 mm to 1.8 mm. Other dimensions of the pin may be modified for example, the cross-sectional shape of the pin may be selected from circular, oblong, square, or flatted shapes. Preferably the electrode pins extend at least 2 mm, more preferably at least 5 mm and more preferably at least 10 mm into the stem tissue. In some embodiments, the pins may extend up to 40 mm within the stem.

The selection of electrode pin shape and depth of penetration of the electrode pin into the stem depends, in part, on the characteristics, such as the diameter, of the stem itself. The pins of deployable sensors of the present invention may be impelled, e.g. pushed by hand, into the stem of a plant, or guide holes may be prepared for the pins. The guide holes 56a to 56d in the stem or trunk 5 of a plant, such as a vine, may be prepared by using a template that has an aperture arrangement identical with the aperture arrangement found on the probe locator through which the electrode pins pass. After guide holes have been prepared, the pins are extended through the locator and inserted into the prepared guide holes. The pins are pushed into the pilot holes until the locator is held in place against the stem by the electrode pins, such as by, for example, pin heads. Alternatively, the sensor including probe and control enclosure containing control circuitry, may be pre-assembled and inserted into the stem as a unit. In some embodiments, the guide hole depth 57a to 57d may be formed to about the same length as required for the length of pin to be insertion into the stem. The pin length may be varied depending on factors such as age and thickness of the stem. In embodiments, the pin length is about 12 mm to about 25 mm, more preferably about 15 mm to about 20 mm. It was found that this length is sufficiently versatile that it is long enough to penetrate tissue beneath bark on older stems but still be short enough that it doesn't pass completely through younger narrower stems such as found with new growth. In embodiments, the guide holes may be slightly shorter than the length of electrode pin required for insertion into the stem, thus allowing for the head of the pin to be driven or pushed into undrilled wood thereby further increasing the strength of the engagement on the electrode pin with the stem wood. The strength of engagement may be further facilitated when the heads of electrode pins with arrow heads are driven into undrilled wood. Preferably, the electrode pins do not extend completely through the stem, that is, do not breach the opposing side of the stem to which the sensor is attached.

Turning to FIG. 5B, the locator may be longitudinally displaced on the stem as indicated by the arrow 61. Alternatively, the locator may be latitudinally aligned on the stem as indicated by the arrow 62. In the embodiment of FIG. 5B, the electrode pin alignment pattern defined by apertures 52a to 52d in locator 22 is linear. However, the electrode pin pattern defined by apertures in the locator may also be offset, scattered or randomised. The apertures 52a to 52b in the probe body have an inner widened region to accommodate the pin head at the proximal end of the corresponding pin, which at least partially retains the pin in the probe body.

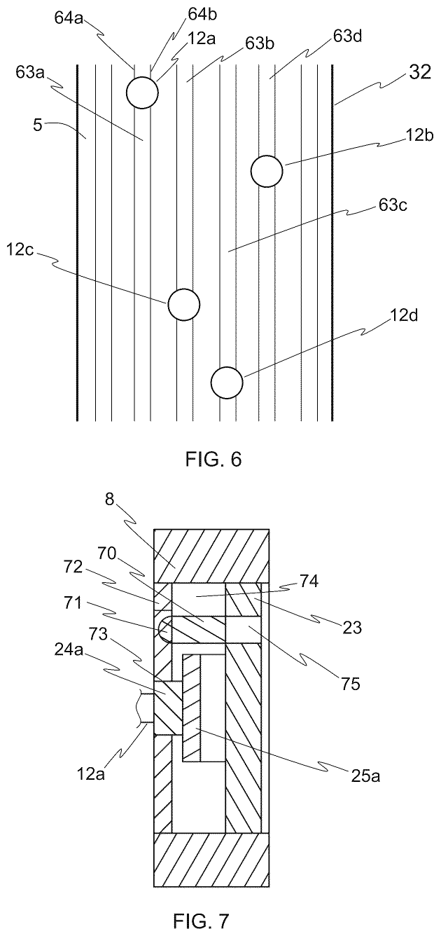

Turning to FIG. 6, an offset pin pattern is illustrated. Cross-sections of electrodes 12a to 12d, represented in the drawing by the indicated circles, are shown as not passing through the same vessel. In the drawing the vessels are denoted by channels 63a to 63d. An example of vessel walls is indicated by 64a and 64b. The applicants have found that in at least some instances alignment of the pins along the same axis as the vessels of the stems may lead to occlusion of vessels such that the fluid connectivity of an electrode pin that is inserted into the same vessel as a pin that is inserted above that pin is reduced due to the occlusion formed by the upper pin. This occlusion effect may have a significant impact on bioimpedance measurements. Employing latitudinal alignment of pins across the stem ensures that the same vessels are not punctured by more than one pin. Similarly, when aligning the locator to extend along the stem, that is in the same direction as the vessels, by off-setting the pins in relation to one another, the occlusion of the same vessel by more than one pin can be avoided.

An example of a pin temperature sensor (pin T-sensor) is provided in FIG. 7 which illustrates a detailed view of the head of an electrode of embodiments of the present invention. The figure illustrates a temperature probe 70 adjacent to the head 24a at the proximal end of electrode 12a (only a partial detail of which is shown). The tip 71 of the pin T-sensor is located within a thermal transfer medium 72 such as a silicone based heatsink compound or thermal adhesive, which is also in contact 73 with the electrode head 24a. The proximal end of the pin including head portion 24a approaches a temperature equilibrium with tissue in the plant stem as a result of the greater thermal mass of the plant tissue relative to the electrode. The high heat transfer properties of the heatsink paste or heatsink adhesive, and the close proximity of the temperature probe tip 71 to the electrode head 24a, ensures that the temperature at the probe tip closely follows the temperature of the electrode head. Connectivity of the pin T-sensor extends through 75 the backplate 23 into the control system to enable temperature data logging. If required, and to obviate overriding external temperature effects, for example, overheating of the control box, an insulating material may be employed in the cavity 74 to thermally isolate the temperature sensor tip and electrode head.

In some embodiments a temperature sensor is located at a tip of electrode pin, or within or along the length of the electrode pin, within the plant stem to measure stem temperature in situ. For example, the electrode may be, cylindrical, that is hollow, with a temperature sensor wire extending along the length of the hollow chamber of the electrode or attached at the tip of the electrode. In certain embodiments, a temperature is located on a separate pin and inserted into the plant tissue in a similar manner as for the impedance electrodes. Suitably, the temperature probe may be directly in contact with the nail head or connector.

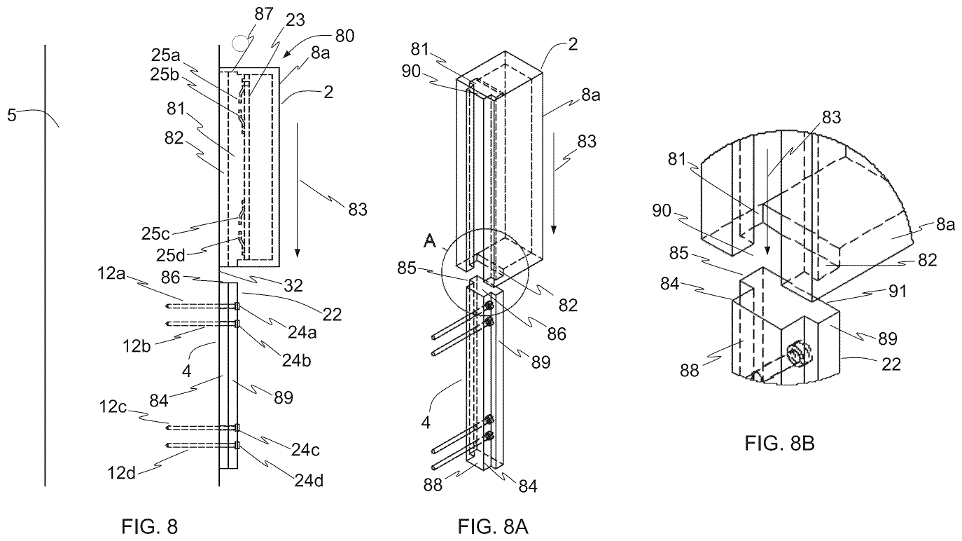

Illustrated in FIGS. 8, 8A and 8B, is an embodiment 80 of a sensor of the present invention wherein the control system enclosure 2 is slidably engageable with the probe 4. Accordingly, the enclosure may be removed from the probe electrodes and locator by the sliding off of the enclosure. This allows access to both the electrodes and the control system circuitry for maintenance and/or replacement of parts. FIG. 8 illustrates a probe 4, including probe body 22 and electrodes 12a to 12d, installed within stem tissue and against the plant stem 5. The heads 24a to 24d of the electrodes are partially disposed within the probe body where the broadened head retains against a shelf within the probe body to at least partially retain the pin and allow the neck of and shaft of the pin to pass therethrough. The pin heads at least partially protruded from the back surface of the probe body 22 in order to contact with the connectors 25a to 25d. In the illustrated embodiment, the probe body is formed with a narrower main body 84 and extended tongue portions 85. The narrow portion 84 and tongue portions 85 and 89 of the probe body mate with respective complementary channels or grooves in a sliding engagement manner, as directionally indicated by arrow 83. The tongues 85 and 89 slide into complementary grooves 81 and 82 and the narrower main body 84 slides into groove 90. The respective tongues and grooves cooperate to retain the upper casing 8a to the probe. An inner wall 87 of the housing 8a acts as a barrier to the travel of control box 2 with respect to probe 4 stopping the housing against upper end 86 of the probe body. When the enclosure 2 has been slid into final position along the probe 4, the stop 87 respectively aligns the electrode heads 24a to 24d with the respective complementary connectors 25a to 25d and the pins are fully retained in place by the connectors attached to the backplate of the upper casing. The base 88 of the probe body is an exposed external face of the sensor and abuts the plant stem when the sensor is in use. Suitably, the connectors 25a to 25d are formed so as to minimise accidental catching of the electrode heads against the connectors during insertion or withdrawal of the probe which could potentially result in irreversible mechanical deformation of either the electrodes or connectors. Moreover, the connector or electrodes may be resilient or sprung such that good contact is ensured between the electrode heads and connectors. When the control box 2 has been slid into position, the respective contacts align to allow transfer of data between the probe and control box. Suitably, the present embodiment is of use for backward compatibility—sensor hardware can be updated, chips replaced, electronics replaced, firmware updated &c, whilst allowing the probe to remain undisturbed within the stem. As further illustrated in FIG. 8B, which is an exploded view of a portion of the embodiment of FIG. 8A, the tongues 85 and 89 slide into grooves 81 and 82 and the narrower main body 84 slides into complementary channel 90 creating a sealing fit between the locating probe body 22 and the casing 8a. The direction of travel of casing 8a is indicated by arrow 83. The probe body has an external face 88 forming an outer surface of the deployable sensor and an inner surface 91.

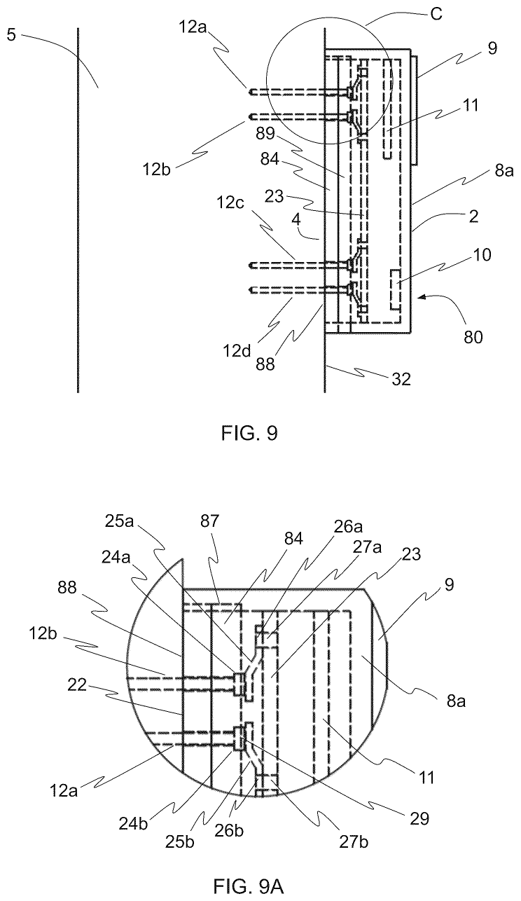

FIG. 9 illustrates a cross-sectional view of the sensor embodiment 80 in situ against stem surface 32 and in stem 5 of the plant, with the pins 12a to 12d inserted into the stem and in contact with plant tissue. The control system including housing 2 is engaged in place and retained on the probe 4. Also illustrated are the rechargeable battery 10, solar panel 9, PCB 11, upper housing 8a, backplate 23, tongue 89, main body 84 and exposed base 88 of the probe body 22. FIG. 9A is a detailed view of the circle section indicated by “C” in FIG. 9. The detailed view illustrates the alignment and contact 29 of the electrode heads 24a and 24b, which are partially retained by locating probe body 22, with the connectors 25a and 25b which are in turn connected to 26a and 26b and in further electrical communication through vias 27a and 27b in backplate 23 with control system circuitry such as PCB 11. The alignment is implemented by the stop 87 which halts the travel of the probe body 22 within the control box 2. The electrode heads are illustrated as raised above the surface of the locator ensuring suitable contact is maintained between the electrode head and connectors for electronic transmission of measurements and other electronic information. The electrode heads are retained in place by the engagement of the upper casing 8a with the locating probe body 22.

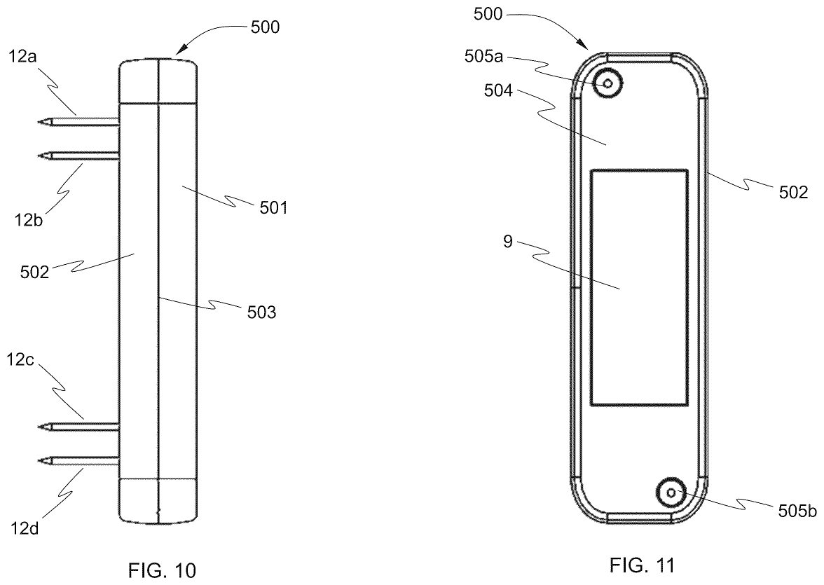

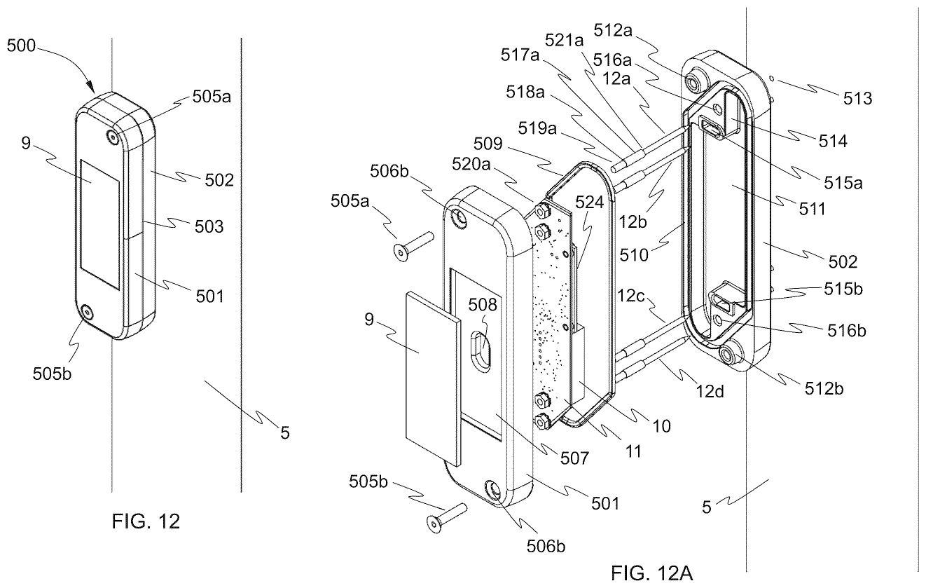

FIG. 10 illustrates side elevation of sensor embodiment 500 of the present invention. The sensor includes electrodes 12a to 12d, and an upper casing or lid 501 and a probe body 502, which mate at seam 503. By securing the upper casing to the probe body, which acts as a base of the field-deployable sensor, a housing for the control system circuitry is formed. FIG. 11 displays a front elevation of sensor embodiment 500 which also reveals a solar panel 9 attached to the surface 504 of the casing 501. Fasteners 505a and 505b retain casings 501 and 502 together at join 503. FIG. 12 is an isometric view of embodiment 500 against a stem 5 of a plant. FIG. 12A is an exploded isometric view of embodiment 500. Displayed in FIG. 12A is recessed region 507 into which fits solar panel 9. The recessing of the solar panel into upper casing 501 further protects the solar panel from breakage, which might occur through corners or edges of the panel being accidentally caught. The solar panel may be held in place with a fixative such as an adhesive. The aperture 508 in upper casing 501 allows for passage of cabling and the like from the solar panel to the control system within the enclosure such as electrical connectivity to PCB 11 and battery 10. Apertures 506a and 506b allows for screws 505a and 505b to pass through the upper casing and engage with raised screw holes 512a and 512b. FIG. 12A displays 4 electrodes 12a to 12d each of which are substantially similar. Ancillary components associated with 12a will be described in additional detail. It is understood that unless stated otherwise similar componentry is associated with each of the electrodes in the illustrated embodiment. The electrode 12a is retained against the PCB 11 by fastening nut 520a. The top portion 519a of electrode 12a passes through the PCB and is threaded to engage with the nut 520a. Nut 520a may be metallic such as stainless steel or may be a plastic such as nylon. The upper face of the PCB down onto which the nut tightened is blank circuit board, that is, circuitry or trace, exposed of otherwise, does not pass beneath the nut, which thus overcomes problems associated with wear through screwing down of the nut. The action of the nut against a trace during tightening of the nut can result in the trace being worn away. The profile of the fastener is also slim to assist with actuation and avoid interference between componentry.

The insertion of the electrodes of the biosensor into the tissue of the stem or trunk of a plant may require application of force to biosensor including the electrodes. Moreover, and as earlier alluded to, when deployed in the field during the growing season, the biosensor may be subject to the rigors of the environment and/or machinery. It is thus desirable that the electrodes are well-retained in the base of the biosensor and that electrical connectivity between the electrodes and control system is maintained. The electrode has an expanded section 517a, of increased cross-sectional area to distal end of the electrode and head, which has both upper and lower shoulders. The upper shoulder 518a is broader than the aperture through the PCB for electrode head 519a and therefore does not pass through the aperture but abuts the underside of the PCB. Located on the underside of the PCB at the junction of the shoulder with the PCB there is a location of electrical connectivity of the electrode with the PCB circuitry, for example, a region of exposed trace. It is the dual action of the screw on the upper side of the PCB and the shoulder on the underside of the PCB that retains the electrode 12a perpendicular to the PCB. As discussed, electrodes 12b, 12c and 12d may be similarly retained.

The lower shoulder 521a of the enlarged diameter portion 517a of the electrode 12a is similarly retained by a shelf inside aperture 516 located in the solid moulded region 514 of base 502. The shelf inside aperture 516a is configured such that the lower narrower portion of the electrode 12a passes through a narrower channel below the shelf and out through the back of base 502 and ultimately into the stem 5 of the plant, such as indicated at location 513 on the stem. The greater cross-sectional area lower shoulder of the 517a portion of electrode 12a is unable to traverse past the shelf and is thus retained thereto against the shelf.

In addition to apertures 516a and 516b in base 502, there are also apertures 515a and 515b which include a well portion. The well portion is formed from an expansion of a section of the aperture and provides a space around part of the proximal shaft end of the electrode. A temperature sensor may be located adjacent the well in contact with a heat transfer media in the well, such as a thermal paste, which is also in contact with the shaft. Accordingly, the well may be a thermal well filled with an electrically non-conductive heat transfer substance to provide a thermal equilibrium between the electrode shaft and the thermal well. In particular, the thermal wells 515a and 515b may be in thermal equilibrium with the stem temperature. The equilibrium established between thermal well 515a and biological tissue within stem 5 where pin 12b is inserted (said pin passing through thermal well 515a) and the thermal equilibrium established between thermal well 515b and the stem tissue in the region wherein pin 12c is disposed, may result in different temperatures in each of the respective thermal wells due to a temperature gradient along the stem. The temperature in each of the thermal wells 515a and 515b may independently change in response to regional changes in stem tissue temperature. Similarly to what is described for apertures 516a and 516b, at the bottom of the thermal well is a narrower channel for passage of the electrode through the base and a shelf to retain the lower shoulder of raised diameter portion of the electrode.

Suitably, at least one pin T-sensor is located adjacent the proximal end of a pin within the biosensor enclosure and said pin T-sensor is in thermal equilibrium with the proximal end of the stem, head or shaft portion of the pin. Preferably, there is an upper pin T-sensor adjacent an upper inner pin and a lower pin T-sensor adjacent a lower inner pin. Suitably, the pin T-sensors are disposed within thermal wells containing a heat transfer media in thermal communication with the proximal end of pins. Suitably, the pins approach thermal equilibrium with the stem temperature. It was found that by electrically isolating the electrode from temperature sensors, but establishing thermal connectivity between the stem, electrode and on-board temperature sensor, that temperature data may be acquired without interfering with the acquisition of stem bioimpedance values. Suitably, the temperature sensor is electrically isolated from the electrode to avoid interference with the acquisition of bioimpedance values from the stem tissue. Suitably, the thermal conductivity of the electrode material is sufficiently high such that there is minimal lag between changes to: environmental temperature, stem temperature; and electrode temperature. That is, the electrode temperature closely follows the stem temperature when the environmental system is in a state of temperature flux, particularly rapid changes in temperature that may be brought about by, for example: the sun rising and setting and weather patterns (for example, in the southern hemisphere, southerly weather changes). Rapid temperature changes that need to be accounted for may occur, for example, when the sunlight first hits the plant stem in the morning.

Suitably, the on-board temperature sensor is thermally isolated, such as with thermal insulation, from the chassis, and from other potential heat sources such as the on-board rechargeable battery.

In further embodiments, not illustrated, a temperature probe may be located within the plant canopy so as to acquire a microclimate temperature within the canopy. And yet again still further embodiments, a temperature probe may be located above the canopy, such as on a trellis post. Suitably, more than one temperature probe may be used to acquire temperature data related to the plant to which is attached a biosensor of the present invention. For example, temperature probes may be located at one or more of: internal to the control box, external to the control box, against the plant stem, within the plant stem, on an electrode with the plant stem, within the plant canopy or above the plant canopy.

Generally speaking, when temperature increases the impedance drops due to increased ion mobility. Accordingly, temperature readings are acquired and logged concomitantly with impedance measurements logged from the stem. The acquisition of accurate stem temperature requires the temperature sensor to be at a temperature equilibrium with the stem. One option is to have the temperature sensor inserted into the stem of the plant adjacent to where impedance values are being acquired. However, a problem with this approach is the potential for interference with acquisition of bioimpedance values, for example, through electrical interference with the signal being acquired.

Also attached to the underside of the PCB is the module 524 for transmitting over long range radio (LoRa) with the system microcontroller also embedded in the same unit. The cavity 511 provides space for components such as the rechargeable battery 10, antenna and the microcontroller/LoRa module 524. In embodiments, the antenna may be integrated into the PCB. The channel 510 is part of the sealing mechanism of the sensor. Upon engagement of the base to the lid a rubber gasket 509 which is displaced within channel 510 provides for sealing engagement between the base and lid. In some embodiments, a complementary mirror channel may be provided on the underside of the lid 501 such that the gasket 510 is at least partially displaced within each channel.

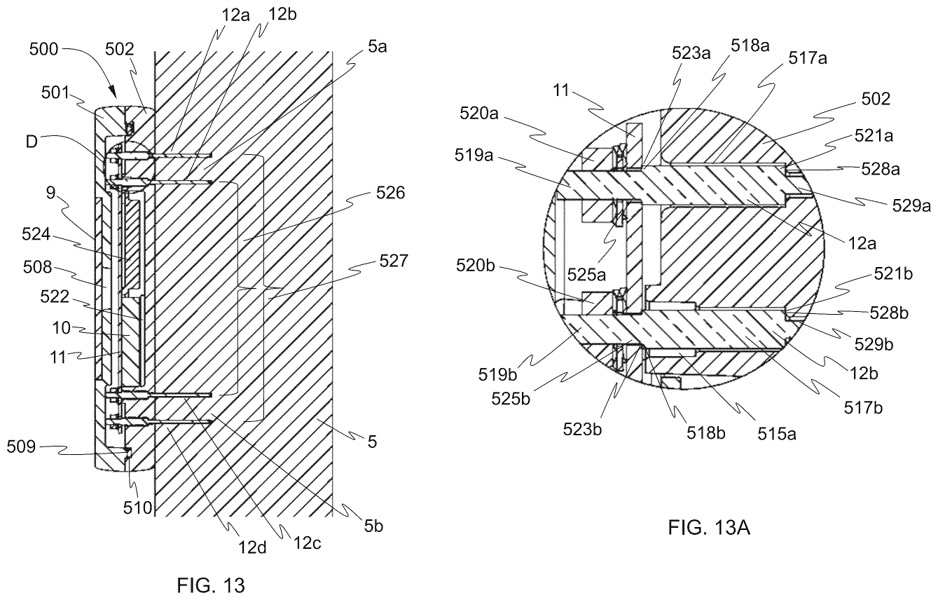

Illustrated in FIG. 13 is a cross-sectional view of embodiment 500 in place against the stem 5 of a plant. Displayed is a solar panel 9 recessed into lid 501 and in communication with the control system via aperture 508. The control system includes microcontroller/LoRa module 524, battery 10 and antenna 522, all of which in the present embodiment are located in a cavity on the underside of PCB 11. The cross-section of the gasket 509 and gasket channel 510 are also indicated in the figure. Illustrated in FIG. 13 is an upper stem tissue zone 5a at a first temperature in the region of upper electrodes 12a and 12b and a lower stem tissue zone 5b in the region of lower electrodes 12c and 12d at a second temperature. Suitably, at least one upper and at least one lower electrode is in thermal communication with a temperature sensor on-board the biosensor such that the temperature in the region of both zones 5a and 5b are separately acquired. The convention of “upper” and “lower” is merely used to differentiate one end of the impedance biosensor from the other as it is understood that the sensor may be attached into the stem bidirectionally, that is either way around. The bracket 526 indicates an inner pair of electrodes 12b and 12c and the bracket 527 indicates an outer pair of electrodes 12a and 12d.

FIG. 13A is an expanded view of a selection of embodiment 500 indicated by the circular region “D” in FIG. 13. Displayed in the expanded view is PCB 11 through which passes the upper threaded portions 519a and 519b of electrodes 12a and 12b respectively. The nuts 520a and 520b retain the respective electrodes in place through cooperation with the respective shoulders 518a and 518b which abut the underside of PCB 11 and are unable to pass through the respective apertures 525a and 525b in PCB 11. The apertures 525a and 525b may be slotted to compensate for intolerances that may occur during the manufacturing process such as when the base is formed or moulded. If there is a slight mis-match of alignment of the electrode heads (retained by the channels in the base) with the apertures in the PCB for the electrode heads, then this can be compensated for by slotting or elongation of the apertures 525a and 525b. The upper shoulders (518a and 518b respectively) contact the underside of the PCB 523a and 523b) through which is achieved electrical communication between the electrode and PCB. The respective lower shoulders 521a and 521b are retained by respective shelves 528a and 528b. Accordingly, the PCB 11 is also retained against the base 502 by the fastening of the electrodes 12a to 12d to the PCB 11. As previously discussed, the lower narrower portions of electrodes 12a and 12b traverse through narrower channels 529a and 529b out through to the other side of base 502 for insertion into a plant stem. The hollow illustrated at 515a is a portion of the thermal well space around electrode 12b. Enclosed within the biosensor casing are moisture sensitive components. Accordingly, the electrode channels which pass through base 502 are sized to snugly fit the electrodes which pass through the channels so as to exclude the ingress of moisture around the electrode, along the channel, and into the enclosure. Suitably, the electrodes may be press-fit into the base. In some embodiments, the moulding of the base of the sensor may be formed or moulded around the electrodes during manufacture, thus retaining the electrodes in the base of the sensor and sealing the electrode shafts within the base.

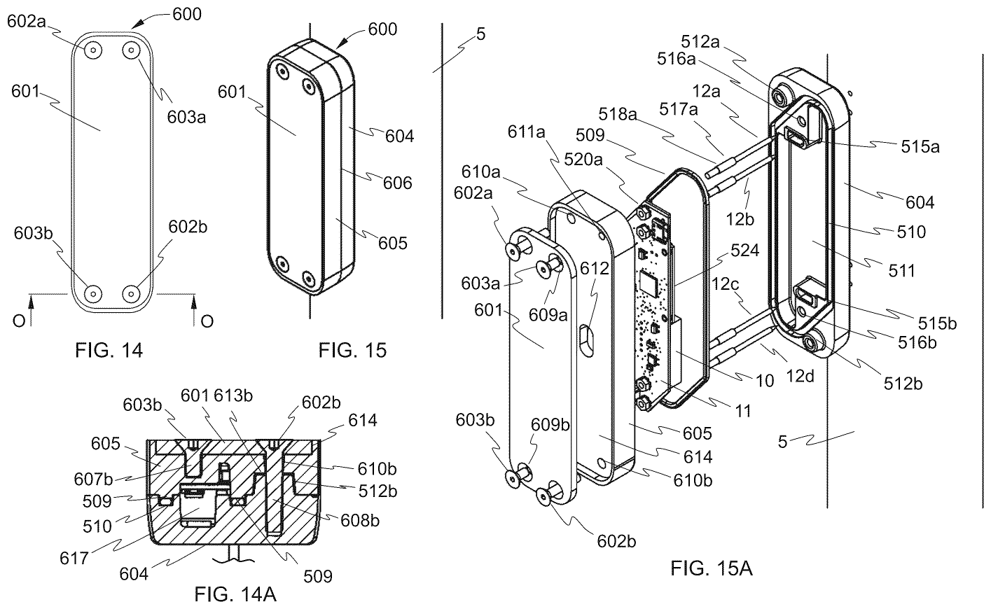

FIG. 14 illustrates a front elevation view of embodiment 600 of the present invention. In FIG. 14 a removable solar panel 601 is displayed substantially covering the entire upper surface of sensor 600. The advantage of solar panel 601 is replaceability in the event of failure and an increased light capture area, which supports battery charging under lower light conditions. The solar panel 601 forms a water and dust resistant seal when attached to upper casing 605. Considering both FIGS. 14 and 15, the solar panel is attached to sensor 600 onto upper casing 605 by fasteners 602a, 602b, 602c and 602d, each of which passes through the solar panel. The sensor 600 is comprised of an upper casing 605 and lower casing or base 604 which cooperate to mate together in a complementary manner, at seam 606, to form a secure housing for the control system circuitry. The lower casing 604 also forms the probe body through which the probe electrodes traverse.

Turning to FIG. 14A which illustrates a view of the cross-section “O-O” as shown in FIG. 14, The fastener 602b engages with threaded hole 607b and retains the solar panel 601 against the upper casing 605 of the enclosure whilst the fastener 603b passes through channel 610b in the upper casing 605 of the enclosure and engages with threaded channel 608b in the base 604 of the enclosure to retain the solar panel against the top 605 of the enclosure and to additionally retain the upper casing 605 against the lower casing 604, with gasket 509 providing a seal between the upper and lower parts of the enclosure thus protecting the internal control system from external environmental conditions such as moisture and dust. The screw mounting hub 512b fits within complementary recess 613b of the top panel 605 as a further measure to secure the top against the base of the enclosure. The solar panel 601 is also recessed 614 into upper casing. Suitably, the cavity 617 adjacent the moulded hub containing the thermal well may be filled with insulating material for further isolation of the thermal well temperature from the cavity 511 temperature.

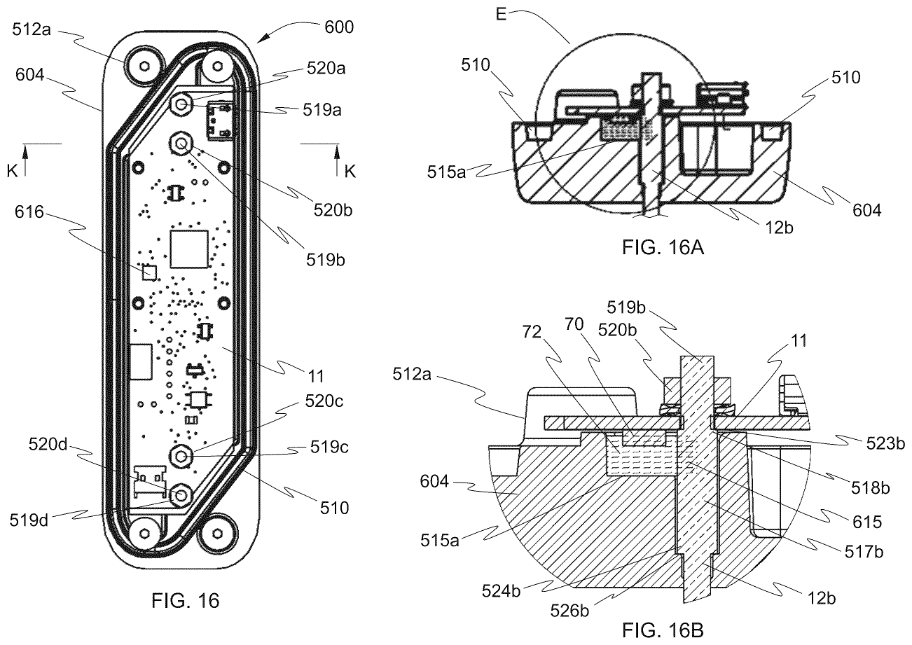

Turning to FIG. 15A, illustrated is an exploded view of the isometric view displayed in FIG. 15. As shown, the fasteners 602b and 603b may have complementary countersunk recesses 609a and 609b with respect to the upper surface of solar panel 601. Suitably, the countersunk recess in the solar panel is reinforced, for example with a countersink lining formed of a metal or plastic, so as to avoid damage to the solar cells such as cracking which might occur when the underside of the screw heads engage with the solar panel upper surface during actuation of the screws. The recessed 614 upper casing 605 has an aperture 612 to allow for connectivity of the solar panel to the internal control system, for example, electrical cabling. Apertures 610a and 610b in the upper casing allow fasteners 602a and 602b to pass through the upper casing and engage with threaded channels in the hubs 512a and 512b of the lower casing 604. On the other hand, fasteners 603a and 603b engage with retaining means in the upper casing 605, for example, fastener 603a engages with complementary channel 611a to secure solar panel 601. By removing fasteners 602a and 602b the upper casing 605 may be disengaged from the lower casing 604, without removal of the solar panel, allowing for access to internal components of the control system, such as for maintenance. Other means of fastening may be employed. Electrodes 12a to 12d extend through bottom casing 604. The uppermost 12a and lower most 12d extend through channels 516a and 516b respectively, whilst the inner electrodes 12b and 12c traverse through channels 515a and 515b which also constitute thermal wells. The electrodes are retained against the PCB 11, for example electrode 12a by nut 520a in a similar manner to as previously described for embodiment 500 of FIG. 13. If desired, at least a portion of the electrodes may be coated in a highly conductive corrosion resistant material such as gold. Although the present embodiment illustrates fastening of the electrodes to the PCB with screws other means of attaching the electrodes to the PCB are envisioned, for example, soldering of the electrodes to the PCB. The PCB 11, is also similarly retained against the bottom plate 602 by the same fastening mechanism as previously described for embodiment 500. Cavity 511 accommodates components of the control system within the controller such as rechargeable battery 10 and the LoRa and microcontroller module 524. The PCB 11 is removable for updating of hardware and software, for example on-board firmware. Considering now FIG. 16, illustrated is a view of embodiment 600 with the upper casing 605 removed exposing the upper surface of PCB 11. Visible are fasteners 520a to 520d which provide a complementary threaded hole to the threaded electrode heads 519a to 519d and which act to both retain the electrodes against PCB 11 and to retain the PCB against base 604. It is known that long cable runs between impedance probe electrodes and data capture hardware within a sensor control system can negatively affect signals being acquired. The attachment of the electrode directly to the control system circuitry, as described herein, can reduce noise and improve signal integrity.

The gasket channel 510 extends around the PCB 11 and control system. FIG. 16A illustrates a cross-sectional view of the embodiment of FIG. 15 at the cross-section “K-K” indicated in FIG. 16, including: gasket channel 510, enclosure base and electrode locating and spacing body 604, thermal well 515a and electrode 12b. FIG. 16B is an expanded view of the region of FIG. 16A, the region indicated by the circle “E”. Illustrated in the figure is the complementary hub 512a which is a moulded portion of base 604 adapted to receive fasteners to retain base 604 against the enclosure top portion. Electrode 12b passes through PCB 11 and is retained by threaded engagement of the top 519b of electrode 12b with fastener 520b. The fastener 520b, along with the shelf 528b and shoulders 518b and 521b, concomitantly acts to secure the electrode to the PCB 11, and the PCB 11 to the base 604. The underside portion 523b adjacent the shoulder 518b provides electrical conductivity between the electrode 12b and the PCB 11 through contact of the shoulder 518b with an exposed portion of conductive trace on the underside of the PCB, thus allowing electrical information to be transferred from the electrodes to the PCB circuitry. On the underside of PCB 11, a temperature sensor 70, which is electrically buffered from the impedance measurement circuitry, is disposed in thermal well 515a and is in contact with the heat transfer medium 72 disposed within thermal well 515a. Suitably, the heat transfer medium is electrically non-conductive and thermally conductive. The heat transfer material may be a solid, a liquid or a paste. Suitably, the heat transfer medium could be a thermally conductive plastic material. It is envisioned, for example, if employing infrared temperature sensing, that the heat transfer material could be metallic such as copper. As such, the well could be occupied by a thermally and electronically conductive metal such as copper metal through which would pass the pin. An infrared T-sensor could register the temperature of the copper body in the well without contacting the body.

Without wishing to be bound by theory, the greater thermal mass of the plant, into which the electrode is inserted, equilibrates the electrode to the plant temperature at the location within the plant at which bioimpedance values are being acquired. This thermal equilibrium is consequently reflected within the thermal well, through contact 615 of the electrode with the heat transfer medium—the temperature of which is recorded by the temperature sensor 70 also disposed therein.

As previously discussed, the expanded region 517b of electrode 12a provides upper and lower shoulders. The region 517b is adapted as: (a) part of the retaining mechanism of electrodes to the PCB 11; (b) part of the retaining mechanism of PCB 11 to the base 604 of the sensor, and (c) part of the mechanism for establishing electrical conductivity between the PCB circuitry and the electrode. However, the enlarged diameter region 517b is also adapted, through its increased diameter, and therefore greater cross-sectional area and mass, to provide a greater thermal mass, relative to the heat transfer medium, to thus promote establishing a temperature equilibrium between the plant, electrode 12b and the heat transfer medium, such that the temperature with the plant adjacent the electrodes where they extend into the plant, is more accurately reflected by the heat transfer medium.

Preferably, the deployable sensor on-board temperature monitoring is comprised of three temperature sensors, a temperature sensor located in a lower thermal well which is electrically isolated but in thermal communication with a lower electrode, and said lower electrode is in thermal equilibrium with a lower stem location into which the electrode is disposed, a temperature sensor located in an upper thermal well which is electrically isolated but in thermal communication with an upper electrode and which is in thermal equilibrium with an upper stem location into which the electrode is disposed, and a third temperature sensor 616 (see FIG. 16) centrally displaced on the PCB which is in thermal equilibrium with at least the enclosure. Suitably, the separate T-sensor mounted to the PCB may be in equilibrium with the enclosure, so the stem temperature can be calculated based on the relative thermal resistance of the system.

The terms upper and lower are relative and refer to location on the stem with respect to the base or origin of the stem (e.g. a branch point or where the trunk emerges from the ground. Typically, lower is basal and upper is apical.

It was unexpectedly identified that longitudinal stem temperature gradients (from lower plant stem to upper plant stem or vice versa) may need to be accounted for when acquiring bioimpedance values across stem tissue.

As previously observed, bioimpedance is temperature dependent and accurate plant temperature measurement is highly desirable in order to acquire accurate bioimpedance values which suitably reflect the plant system and greater environmental conditions. As such, a temperature reading is preferably acquired along with bioimpedance data in a time dependent manner. Accordingly, stem temperatures form part of a discrete plant data package including: unique sensor identification, location, temperature, log time, and bioelectrical information including impedance. This data package may then be correlated with other regional or local data, for example weather data including temperature, precipitation, irradiation and evapotranspiration. Typically, multiple sensors may be deployed across a number of plants in an orchard, vineyard or field, for example in a grid formation. Each sensor that is deployed has a unique digital signature or sensor identifier which allows for identification of data origin during transmission events-differentiating the information packets sent from each sensor. The unique identifier also allows for identification of the geospatial location of the sensor, which could be logged, for example, when the sensor is deployed in the field and then attached to the unique sensor identification.

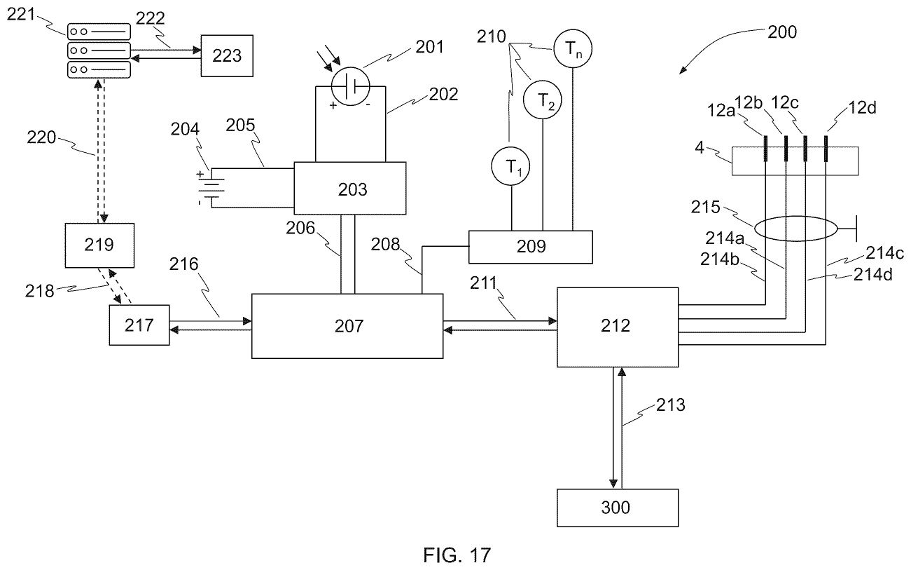

FIG. 17 is a schematic representation of a data collection and transmission system 200 of the present invention. Solar cells 201 connected to a solar controller 202 provides charge for system battery 204. The system battery may be any suitable rechargeable battery such as a single cell lithium-based battery. In one example, the battery is a 3.7V battery (4.2V at maximum charge) that is regulated by a voltage regulator to provide a control system voltage of about 3.3V. The system microcontroller 207 loads battery 204 through the solar controller 203. Suitably, the solar controller and microcontroller may be integrated on the same printed circuit board within the control system. In embodiments, where a sensor may be located in a high-density canopy with limited light penetration, such as a kiwifruit vine canopy, a solar panel, electrically connected to the on-board sensor rechargeable battery, may be located remotely from sensor above the canopy to allow for greater sunlight exposure. Operations of the system microcontroller 207 include: programming the analogue front end (AFE) 212, storing data, controlling the calibration sequence 300 and calibration frequency, and transmitting data to a long-range transceiver 219 via communication chip 217. Suitably, the calibration system includes one or more high precision resistors from which to derive impedance values for calibration. Temperature data accumulation 209 is also transmitted 208 to the microcontroller from temperature probes T₁, T₂ . . . Tₙ indicated by the reference numeral 210. As discussed, the microcontroller 207 communicates with the AFE 212 which is in electrical connectivity 214a to 214d with electrodes 12a to 12d which when the sensor is in operation are located within the stem of a plant. Suitable AFEs are the AD5940, AFE4500, AD5941 or MAX30002 chips. The electrical connections 214a to 214d are shielded to minimise disruptive signal interferences. Suitably, the shield 215 is connected to a common return path for current from the different components of the control circuitry. The shield may be, for example, connected to the power ground, the analogue ground, which is at the same potential as the power ground but kept separate to reduce noise from digital circuitry, or a virtual ground at an elevation above zero volts, which is essentially the virtual ground around which the output signal oscillates.

The microcontroller can communicate with a LoRa transceiver with hardwired digital communication for example, an SPI bus. LoRa may be built into a microcontroller module or added as a separate component depending on requirements. Associated with the microcontroller may also be a Bluetooth. The Bluetooth may be used, for example, for device setup, firmware updates, or suitably other tasks.

The communication chip 217 transfers 218 data from the microcontroller 207 to the gateway 219. A suitable gateway is a LoRa gateway. The gateway may also be, for example, a third-party gateway. Data is then transferred 220 to a server 221 via a range of suitable communications means. Suitable communication means include: cellular, WI-FI, or ethernet. The uploaded data is processed and then provided 222 for access to a user interface 223.

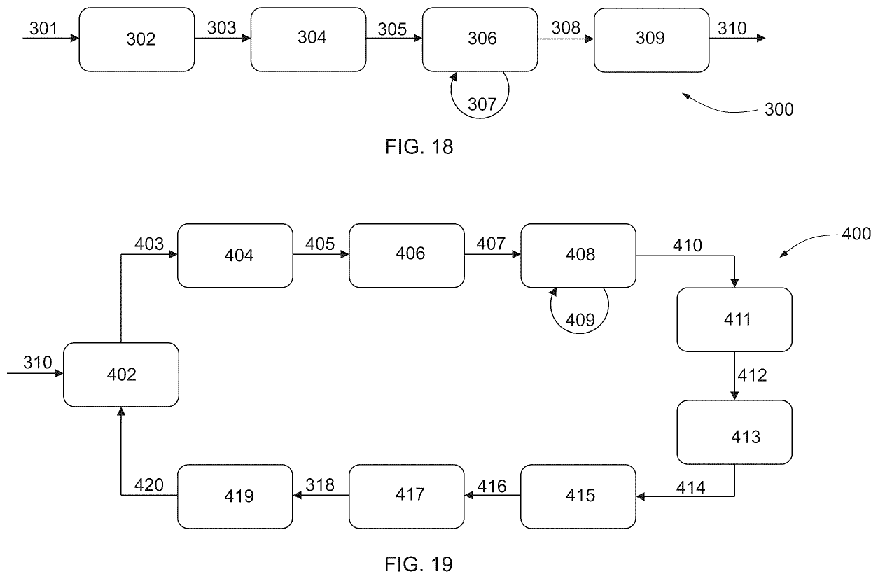

FIG. 18 illustrates an exemplary calibration sequence 300 of the present invention. The control system is powered on 301 and a command is sent to commence the calibration sequence. This initiates 303 a switch 304 to the calibration circuit. Subsequently 305, the calibration sequence 306 is run including a repeat 307 of the sequence for set frequencies. Following 308 running of the calibration sequence the calibration data is saved to memory 309 for system reference. The measurement sequence is then subsequently commenced 310.

FIG. 19 illustrates a measurement sequence 400 of the present invention. Commencement initiated from the calibration sequence 310 is followed by a controller wake up 402. The controller subsequently 403 sends a command to commence measurement 404 and ensuing 405 is a switch to the plant tissue output 406. Subsequently 407 measurements are run 408 with a repeat 409 for set frequencies. The data is then subsequently 410 sent to the microcontroller which is then adjusted 413 based on the saved calibration data. Following 414 tissue impedance data acquisition temperature readings are taken 415. Following 416 unique sensor identification, temperature data acquisition, a data package including date, time, temperature and impedance data is sent 417 via long range transceiver for ultimate upload to a server. Following the transfer, the system goes to sleep 419. After a programmed period 420, for example 10 minutes, the controller wakes up and the measurement sequence is repeated.

### EXAMPLES

### Example 1: Exemplary Bioimpedance Acquisition System

An exemplary bioimpedance acquisition system of the present invention includes the following components:
(1) biosensor including:
   (i) probe;
   (ii) control box; and
   (iii) transmission means between the probe and control box;
(2) gateway;
(3) server; and
(4) application.

In the present example the probes are approximately finger-sized units which have 2 or more protruding electrodes that are inserted into the trunk of a woody plant such as a vine or tree. The probes, together with the circuitry enclosed in the protective housing which is in electrical communication with the probe, collect data from the woody plant and wirelessly transmit the data locally to a gateway. To insert the electrode pins into the trunk, holes are drilled in the trunk using a drill blank. The units are intended to be low power such that they can be installed on the trunk and remain there up to 10+ years.

The biosensor apparatus of this exemplary system has the following hardware:
2 or more small electrodes on a probe wherein the probe is connected by a shielded electrical cable to circuitry contained in a protective enclosure;
Custom protective enclosure within which to mount the biosensor control circuitry;
One or more thermocouples or temperature sensors. The thermocouple could be located, for example, inside an additional hole made in the stem, insulated against the trunk, or on the head of the electrode inside the unit;
A 3.7V Li-ion battery;
A solar panel located on the protective housing;
An AD5940 bioimpedance chip for applying electrical AC signals through the electrodes at varying frequencies (10 Hz-200 kHz) and recording the resultant real and imaginary components of the impedance through the plant cell structure;
A low power Bluetooth (BLE protocol) chip;
Communication chip such as a LoRa chip, and
A microcontroller for programming the AD5940, storing data, controlling the calibration sequence and calibration frequency, and transmitting data to a long-range transceiver via communication chip.

### Gateway

A Gateway is a standard product used for LoRa communication and is equivalent to a modem for Wi-Fi. The Gateways should be placed as high as reasonably possible to maximise coverage. The Gateways receive data from the long-range transceivers within a radius of 1-10 km and then upload the data to a server via whatever communications means is most convenient (for example: cellular, WI-FI, or ethernet).

### Server

The server is the back end where the raw data is received from the gateway, tabulated, and converted, using biocircuitry models including physiological understanding of bio-electric systems, into meaningful information about individual plant status (for example: water potential).

### Application

The application is the user interface that displays plant's health and location on the orchard/vineyard. This interface is available, for example, as a client portal wherein access to various hierarchical tiers of the plant health data are provided. The portal may be accessed, for example, by consultants who can view all their customers' installations, through to orchard managers who oversee a specific orchard. Data may be presented via an overlay of satellite imagery of the orchard with pins to each of the units, segregated via irrigation blocks and spatial overlays of the entire orchard through NDVI or other remotely-sensed relative zoning information. The data could also be made available to growers' own applications via an API (application programming interface).

### Example 2: Exemplary Biosensor

An exemplary biosensor of the present invention incorporates the following components:
A control box (including a protective housing) containing biosensor circuitry including the control and communication circuits, plus battery and solar. The box is attachable to a stem of the plant either together with the probe or separately.
In the present example, the communication and main microcontroller contained in the control box (comprising part of the biosensor circuitry), is a standard part from RAKWireless;
A custom designed PCB is attached to the RAKWireless standard part. The custom PCB incorporates the system for temperature measurement, calibration and the impedance circuitry;
The impedance measurement is based on an AD5940 or AD5941 chip which is incorporated into the custom PCB circuit although other suitable chips will be known to those skilled in the art;
A separate probe is connected with shielded wire to the control box. When inserted into the stem of a plant, the electrodes hold the probe in place against the stem of the plant. The probe is comprised of a locating and pin spacing probe body with apertures for electrode pins. The locating body may also be used to adjust the depth of the electrode pins into the stem, depending, for example, on stem thickness. The electrode pins are in electrical communication with the control box via a shielded signal cable. A four-pin electrode measurement system is used to eliminate the error caused by impedance in the probes and at the interface between the electrode and the tissue;
A temperature sensor is located on the custom PCB as well as at least one external waterproof temperature sensor;
In the present example, all four electrode pins are formed from 1.6 mm stainless steel nails. The length of the electrode pins may vary but is typically between 5 mm-40 mm;
Spacing between electrode pins is governed by the pattern of apertures on the probe body and may be from 5 mm-40 mm between an electrode pair. Under a four-pin system there is an outer pair and inner pair of electrodes. The spacing between the inner pair of electrodes is of greater significance as it is between these pair of electrodes that impedance is measured.

### Specifications

An exemplary biosensor has the following specifications:

| SPECIFICATION | VALUE |
|---|---|
| Sampling rate | 0.5 seconds to 24 hr intervals dependent |
| Frequencies | Up to 1000 total between 10 Hz and 200 kHz |
| measured | (option to have equal or logarithmic spacing) |
| Chip used | AD5940 or AD5941 |
| Plants measured | 1 plant per sensor |
| Data collection | LoRa communication to gateway. Forwarding |
| type | to AWS |
| Other data | Temperature on PCB and waterproof external |
| | sensors, stem tissue temperature |
| Probes | 4 × 1.6 mm stainless steel nails in stem. Wire |
| | crimped to nail heads and nail heads housed in |
| | epoxy. |

Sampling rate is selected based on required sampling frequency, energy consumption and limitations of the communication protocol. The data collection type allows for data coming back in real time and was selected on the basis of range to power performance. Stainless steel electrodes were selected for their anticipated stability in ionic fluid (e.g. plant sap).

In some embodiments, the sensor control box may be used as nexus for additional sensor data acquisition, for example, to estimate evapotranspiration. Additional sensors may include: soil moisture probes, wind speed and humidity. Furthermore, light incidence values may, for example, be calculated from solar panel current.

Optionally, the sensor includes a processing means for processing some or all of the raw data onboard the sensor. An example of this type of onboard computation is known as edge computing. The on-board processing could include, for example, deriving the electrical parameters of a representative electrical circuit. Processing could also involve least-squares computation, algorithm processing, machine learning/AI, or the combination of environmental data with impedance data. For example, the impedance data could be adjusted onboard based on the temperature of the plant stem. Suitably, the on-board processing significantly reduces the size of the data packet and provides a range of benefits, including: reduced power consumption and more flexibility for communication. Local storage, such as an SD card, may also be used for saving the raw impedance data on the sensor for manual collection at a future date. Local storage may also be used to save data in case of a communication system problem, whereby the data is then transmittable when the communication system comes back online.

Optionally, multiple sensors are capable of communicating locally with short range communication means, such as Bluetooth Low Energy (BLE) or Bluetooth Mesh, to collate and process data before sending to a gateway. Furthermore, in some embodiments the sensor may communicate locally with third-party devices, such as irrigation controllers, weather stations, or smartphones, to share information locally or integrate this information with the sensor data as part of the onboard processing.

### Example: Deployable Sensor Temperature Calibration

The temperature calibration method uses the thermal dynamics of a manufactured sensor to set coefficients that allow the sensor to accurately derive the temperature of the plant sap and/or tissue once installed in a plant stem. The water bath is assumed to be a proxy for plant sap and/or tissue when the sensor is installed. Calibration steps include the following:
1. Set up the sensor in a controlled temperature room (for example, 20 deg C.), with the electrodes placed in fluid (eg water) at controlled temperature (eg 0 deg C.) to a controlled depth (eg 10 mm for each electrode).
2. Wait for the sensor to reach thermal equilibrium (eg 1 hour).
3. Take a reading of the temperature of the control system (eg with centrally mounted PCB sensor) and temperature readings of the electrode/s (eg with the sensor/s situated in the thermal well/s).
4. Calibrate the coefficients of an equation defining the thermal resistance of the parts of the system to allow accurate measurement of the fluid surrounding the electrodes, which when installed in a plant would be the plant sap.
5. The coefficients could be stored onboard the sensor in the control system to calculate sap temperature at the point of measurement or could be stored in the cloud to calibrate after the temperature data is communicated.

The temperature of the electrode pin could be optionally measured using a thermal well with direct contact with a thermally conductive material (eg heat sink paste) or could be measured with an infrared sensor or alternative approach. For example, an infrared sensor could be disposed adjacent or within the well and directly measure the proximal pin temperature through the well space.

It is a requirement to measure the temperature of the plant sap to compensate the influence of temperature on impedance values. It is advantageous to avoid creating an extra hole in the plant for measuring temperature (eg with a separate thermocouple implanted in the tissue) as it increases the complexity of installation, may interfere with the tissue activity, and an additional incision into tissue increases the risk of disease for the plant. A temperature measurement on the surface of the plant (eg with a sensor on the underside of the probe housing or an infrared measurement of the stem surface) is an alternative, but has been observed to provide a less accurate representation of sap temperature. A measurement that can physically measure temperature within the plant of plant sap and tissue, without additional injury to the plant, is therefore advantageous.

In compliance with the statute, the invention has been described in language more or less specific to structural or methodical features. The term “comprises” and its variations, such as “comprising” and “comprised of” is used throughout in an inclusive sense and not to the exclusion of any additional features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

1. A field-deployable sensor attachable to plants for acquisition of plant-related data comprising:
(a) a probe including;
(i) a probe body with at least one aperture therethrough which locate the placement and spacing of one or more thermally conductive pins within the tissue of a plant;
(ii) one or more thermally conductive pins having distal and proximal ends, said distal end traversing through said aperture in the probe body and insertable into the tissue of the plant, and said proximal end located within the deployable sensor; such that when inserted into the tissue the pin approaches thermal equilibrium with the temperature of the region of the tissue in which the pin is located; and
(b) a temperature sensor (T-sensor) enclosed within the deployable sensor located adjacent the proximal end of the pin to acquire the temperature of the proximal end of the pin;
such that the T-sensor through the thermal equilibration of the pin with the plant tissue acquires plant tissue temperature data.

2. (canceled)

3. The deployable sensor according to claim 1 wherein the at least one aperture further comprises an expanded section forming a well around the thermally conductive pin.

4. The deployable sensor according to claim 3 wherein the T-sensor is situated within the deployable sensor to record the temperature within the well.

5. The deployable sensor according to claim 3 wherein the well contains a heat transfer media such that when the one or more pins are inserted into plant tissue, heat transfer between the tissue, pins and T-sensor establish a thermal equilibrium.

6. The deployable sensor according to claim 5 wherein changes in the thermal equilibrium in response to changing environmental conditions affecting plant tissue temperature are recordable.

7. The deployable sensor according to claim 1 wherein the sensor comprises upper and lower thermally conductive pins each with independent T-sensors such that temperature gradients across the tissue into which the respective pins are located are acquirable.

8. The deployable sensor according to claim 1 wherein the one or more thermally conductive pins also comprise one or more electrically conductive electrodes.

9. The deployable sensor according to claim 8 wherein the sensor further comprises:
(a) an electrical communication means for transmitting electrical data between electrodes and a control system whereby the electrical communication means provides electrical continuity between the electrodes and the circuitry of the control system; and
(b) a control system housed within a protective enclosure, including:
(i) a printed circuit board (PCB) containing control system circuitry;
(ii) an analogue front-end for transmitting excitation signals to the electrodes and for converting electrical data received from the electrodes into electrical impedance values; and
(iii) a microcontroller for programming the analogue front end, and for storing and transmitting data.

10. The deployable sensor according to claim 9 further comprising a T-sensor on the PCB for acquiring temperature data within the deployable sensor in the region of the PCB located T-sensor.

11. The deployable sensor according to claim 9 wherein the sensor comprises a first spaced pair of electrodes.

12. The deployable sensor according to claim 11 wherein the sensor comprises a second spaced pair of electrodes.

13. (canceled)

14. (canceled)

15. (canceled)

16. The deployable sensor according to claim 12 wherein the spacing between the second pair of electrodes is from about 20 mm to about 80 mm, more preferably from about 40-70 mm and the spacing between the first pair of electrodes is from about 60 mm to about 100 mm.

17. The deployable sensor according to claim 9 wherein the pin shafts comprise an enlarged portion with a lower shoulder that engages with and is retained by a shelf in the probe body, and an upper shoulder which engages with and is retained against the PCB said upper engagement forming electrical communication between the pin and the control system circuitry.

18. The deployable sensor according to claim 17 wherein the engaging of an electrode shaft with the PCB secures the PCB and the electrode to the probe body.

19. The deployable sensor according to claim 9 wherein the protective housing comprises a base and an upper casing.

20. (canceled)

21. (canceled)

22. (canceled)

23. (canceled)

24. The sensor according to claim 1 wherein the one or more pins have a diameter in the range from about 500 mm to about 2 mm, preferably from about 1 mm to about 2 mm.

25. A method for acquiring plant data with a field-deployable sensor attachable to plants comprising:
attaching a probe body of the sensor to the plant by inserting one or more thermally conductive pins into the plant tissue wherein said pins traverse through the probe body and are spaced, relatively located, and retained by the probe body;
allowing the pins to approach thermal equilibrium with the plant tissue;
acquiring stem temperature data with a temperature sensor located adjacent a proximal end of a pin in the probe body,
wherein said proximal end of the pin approaches thermal equilibrium with the stem tissue.

26. The method of claim 25 wherein the pins are electrodes and are in electrical communication with a control system housed in a protective casing said control system including: a printed circuit board; an analogue front-end for transmitting excitation signals to the probe and for converting electrical data received from the probe into electrical impedance values; and a microcontroller for programming the analogue front end, and for storing and transmitting data; and wherein the method further comprises the steps of
transmitting an electrical excitation signal to the vascular plant tissue through a pair of electrodes; and
acquiring frequency specific electrical impedance values from the plant tissue.

27. The method according to claim 26 wherein the pins are inserted into the tissue of the plant so as to contact extracellular fluid, vascular tissue and ground tissue of the plant.

28. The method according to claim 27 wherein the electrodes comprise a first spaced outer pair and a second spaced inner pair of electrodes and the method further comprises the steps of:
a timed application of a current signal to the tissue with the first spaced pair of electrodes with the analogue front-end;
simultaneous measurement of the voltage across the tissue with the second spaced pair of electrodes;
simultaneous recording of the temperature in the thermal well with a temperature sensor; and
acquisition and local storing of the resultant data by the microprocessor.

29. (canceled)

30. (canceled)

31. (canceled)

32. (canceled)
